(19) United States
(12) Patent Application Publication
(10) Pub. No.: US 2025/0090498 A1
(43) Pub. Date: Mar. 20, 2025

(54) TREATMENT OF PRIMARY CTLA-4 CHECKPOINT RELATED IMMUNODEFICIENCIES

(71) Applicant: ADIENNE PHARMA & BIOTECH SA, Lugano (CH)

(72) Inventors: Antonio Francesco DI NARO, Morcote (CH); Stefano GIOVAGNOLI, Perugia PG (IT); Matteo PUCCETTI, Perugia PG (IT); Marilena PARIANO, Perugia PG (IT)

(73) Assignee: ADIENNE PHARMA & BIOTECH SA, Lugano (CH)

(21) Appl. No.: 18/746,743

(22) Filed: Jun. 18, 2024

Related U.S. Application Data
(60) Provisional application No. 63/509,223, filed on Jun. 20, 2023.

(51) Int. Cl. A61K 31/404; A61K 9/00; A61K 47/30; A61P 37/02

(52) U.S. Cl. CPC A61K 31/404; A61K 9/0053; A61K 47/30; A61P 37/02

(57) ABSTRACT

The present disclosure is directed to a method of treating a CTLA-4 checkpoint related immunodeficiency by administering a therapeutically effective amount of a pharmaceutical composition comprising 1H-indole-3-carboxaldehyde (3-IAld) or 1-methylindole-3-carboxylic acid.

### CROSS-REFERENCE TO RELATED APPLICATIONS

This application claims the priority benefit of U.S. Provisional Application No. 63/509,223, filed on Jun. 20, 2023, which is incorporated herein by reference in its entirety.

### FIELD

The present disclosure relates to a method for treating a CTLA-4 checkpoint related immunodeficiency disorder in a patient.

### BACKGROUND

Primary CTLA-4 checkpoint related immunodeficiencies are characterized by variable combination of enteropathy, hypo-gammaglobulinemia, recurrent respiratory infections, granulomatous lymphocytic interstitial lung disease, lymphocytic infiltration of non-lymphoid organs (intestine, lung, brain, bone marrow, and kidney), autoimmune thrombocytopenia or neutropenia, autoimmune haemolytic anaemia and lymphadenopathy. Heterozygous CTLA4 mutations in humans are associated with a severe immunoregulatory disorder, which is known as CTLA-4 haploinsufficiency with autoimmune infiltration (CHAI) disease. Another type of primary CTLA-4 checkpoint related immunodeficiencies has been reported in literature, in patients harboring biallelic mutations of the “lipopolysaccharide-responsive beige-like anchor (LRBA)” gene. As the LRBA deficiency also causes a secondary loss of CTLA-4, this recessive disease is called” LRBA deficiency with autoantibodies, T-reg cell defects, autoimmune infiltration and enteropathy (LATAIE).

At present, hematopoietic stem cell transplantation (HSCT) is the only cure for patients with primary CTLA-4 checkpoint related immunodeficiencies. All other proposed therapies are still largely unsatisfactory with the exception of immunoglobulin replacement therapy to correct hypogammaglobulinemia and prevent infections.

Systemic corticosteroids alleviated gastrointestinal symptoms in some patients, but only temporarily.

A treatment with abatacept or sirolimus is recommended to control lymphocyte activation in all patients with CTLA-4-associated CNS syndrome. As recommended for other demyelinating CNS disorders, high dose steroids are still suggested as first-line treatment. If no rapid clinical response is observed, adding high dose IVIG appeared to be valuable. As a second-line strategy, rituximab or cyclophosphamide should be considered while continuing the first-line therapy, as recommended for autoimmune encephalitis.

In conclusion, systemic immunosuppressants and abatacept may provide partial control but require ongoing administration. Despite a significant risk of treatment-related mortality, allogeneic hematopoietic stem cell transplantation offers a possible cure for patients with CTLA-4 insufficiency and eligible for this approach. Another important issue is the need of multiple changes of immunosuppressants owing to adverse effects or steroid dependence. Nevertheless, a therapy is required as untreated or insufficiently treated patients develop further CTLA-4-related symptoms, suggesting a progressive natural course of the disease.

Further, despite the great success of cancer immunotherapy using immune checkpoint inhibitors, their therapeutic benefits are limited by either various resistance mechanisms (Schoenfeld et al, 2020) or the associated toxic effects, including frequent gastrointestinal, endocrine, and dermatologic toxicities and fatal neurotoxicity and cardiotoxicity (Choi et al, 2020). Thus, novel therapeutic strategies that provide manageable side effects to existing immunotherapy would enhance and expand their therapeutic efficacy and application.

### BRIEF SUMMARY

The present disclosure provides, at least in part a method of treating a CTLA-4 checkpoint related immunodeficiency in a patient comprising administering a therapeutically effective amount of a pharmaceutical composition comprising 1H-indole-3-carboxaldehyde (3-IAld) to the patient in need thereof. In some aspects, the CTLA-4 checkpoint related immunodeficiency is selected from the group consisting of: CTLA-4 haploinsufficiency with autoimmune infiltration (CHAI), lipopolysaccharide-responsive beige-like anchor (LRBA) deficiency (LATAIE), a regulatory T (Treg) cell defect, autoimmune infiltration, enteropathy disease, gut inflammation, immune-mediated colitis, gastrointestinal disorder, and gastric atrophy. In another aspect, the CTLA-4 checkpoint related immunodeficiency is immune-mediated colitis.

In one aspect, the pharmaceutical composition comprises a pharmaceutically acceptable carrier. In another aspect, the pharmaceutically acceptable carrier is at least one polymer. In another aspect, the pharmaceutically acceptable carrier is a set of polymers. In another aspect, the set of polymers are Eudragit® polymers. In another aspect, the pharmaceutical composition is formulated for enteric delivery. In another aspect, the pharmaceutical composition is orally administered. In another aspect, the pharmaceutical composition is the form of a capsule, tablet, gel tablet, gel capsule, gel, liquid, or gummy. In another aspect, the pharmaceutical composition is in a tablet or capsule form.

In one aspect, the pharmaceutical composition is administered at an interval of every other day (q.o.d). In another aspect, the pharmaceutical composition is administered at a 3-IAld dosage of at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, least about 16 mg/kg, at least about 17 mg/kg, or least about 18 mg/kg. In another aspect, the 3-IAld dosage is about 18 mg/kg.

The present disclosure additionally provides, at least in part a method of treating a CTLA-4 checkpoint related immunodeficiency in a patient comprising administering a therapeutically effective amount of a pharmaceutical composition comprising 1-methylindole-3-carboxylic acid to the patient in need thereof. In some aspects, the CTLA-4 checkpoint related immunodeficiency is selected from the group consisting of: CTLA-4 haploinsufficiency with autoimmune infiltration (CHAI), lipopolysaccharide-responsive beige-like anchor (LRBA) deficiency (LATAIE), a regulatory T (Treg) cell defect, autoimmune infiltration, enteropathy disease, gut inflammation, immune-mediated colitis, gastrointestinal disorder, and gastric atrophy. In another aspect, the CTLA-4 checkpoint related immunodeficiency is immune-mediated colitis.

In one aspect, the pharmaceutical composition comprises a pharmaceutically acceptable carrier. In another aspect, the pharmaceutically acceptable carrier is at least one polymer. In another aspect, the pharmaceutically acceptable carrier is a set of polymers. In another aspect, the set of polymers are Eudragit® polymers. In another aspect, the pharmaceutical composition is formulated for enteric delivery. In another aspect, the pharmaceutical composition is orally administered. In another aspect, the pharmaceutical composition is the form of a capsule, tablet, gel tablet, gel capsule, gel, liquid, or gummy. In another aspect, the pharmaceutical composition is in a tablet or capsule form.

### BRIEF DESCRIPTION OF THE DRAWINGS/FIGURES

FIG. 1 is a representation of the chemical structure of 1H-Indole-3-carboxaldehyde, also known as indole-3-aldehyde or 3-formylindole (3-IAld, MF: C9H7NO, IUPAC name: 1H-indole-3-carbaldehyde).

FIGS. 2A-2H are graphical representations and photograph images showing that 3-IAld protects from inflammatory pathology in DSS+anti-CTLA-4-induced colitis. C57BL/6 mice were treated with DSS in drinking water for 1 week followed by a recovery period of another week and administered 100 μg of anti-CTLA-4 mAb and 3-IAld as depicted in the experimental schedule (FIG. 2A). Mice were evaluated (FIG. 2B) for % survival, (FIG. 2C) % weight change, (FIG. 2D) disease activity index, (FIG. 2E) rectal bleeding, (FIG. 2F) colon histology via PAS staining, (FIG. 2G) histology score, and (FIG. 2H) epithelial barrier function (ZO-1 and Ki-67 staining). Photographs were taken with a high-resolution microscope, ×20 magnification (scale bars, 200 μm). White arrow indicates rectal bleeding. Yellow arrows indicate inflammatory cells recruitment. Each in vivo experiment includes four to six mice per group (16-24 mice in each experiment). Data are represented as mean±SEM. H₂O, untreated mice. ***P<0.001, ****P<0.0001.

FIGS. 3A-3H are graphical representations showing that 3-IAld provides epithelial barrier integrity and promotes an anti-inflammatory state in murine colitis. C57BL/6 mice, subjected to DSS plus anti-CTLA-4-induced colitis and administered 3-IAld as described in FIG. 2, were evaluated for (FIG. 3A) markers of epithelial functioning; (FIG. 3B) Dextran-FITC and (FIG. 3C) sCD14 levels in the serum; (FIG. 3D) cytokine and (FIG. 3E) calprotectin levels in colon homogenates and (FIGS. 3F, 3G, 3H) AhR-dependent gene expression. Data are represented as mean±SEM. H₂O, untreated mice. *P<0.05, **P<0.01, ***P<0.001. ns, not significant.

FIGS. 4A-4F are graphical representations and photograph images showing that 3-IAld protects mice from immune-mediated colitis. NSG mice were infused with hPBMCs and treated with anti-CTLA-4 mAb and 3-IAld. Mice were sacrificed at 21 days and evaluated for (FIG. 4A) % survival, (FIG. 4B) % weight change, (FIG. 4C) colon histology (periodic acid-Schiff staining) and (FIG. 4D) histology score, (FIG. 4E) ZO-1 protein expression and (FIG. 4F) inflammatory cytokine expression. Photographs were taken with a high-resolution microscope (Olympus BX51), ×20 magnification (scale bars, 200 μm). Each in vivo experiment includes four to six mice per group (16-24 mice in each experiment). Data are represented as mean±SEM. *P<0.05, **P<0.01, ****P<0.0001. Two-way analysis of variance, Bonferroni post hoc test. H₂O, untreated mice.

FIGS. 5A-5D are graphical representations and photograph images of how 3-IAld limits progression of immune-mediated colitis in RAG1-deficient mice. Rag1^{-/-} mice infused with CD4⁺ T cells were treated with αCTLA-4 mAb and 3-IAld. Mice were sacrificed at 21 day and evaluated for (FIG. 5A) % weight change, (FIG. 5B) gross histology, (FIG. 5C) histology score and (FIG. 5D) colon histology (periodic acid-schiff staining). Photographs were taken with a high-resolution microscope, 20× magnification (scale bars, 200 m). For histology, data are representative of two independent experiments. Each in vivo experiment includes 4 mice per group (16 mice in each experiment). Data are represented as mean±SEM. **p<0.01, ****p<0.0001. Two-way ANOVA, Bonferroni post hoc test. H₂O, untreated mice.

FIGS. 6A-6C are graphical representations and photograph images showing how 3-IAld protects from DSS+anti-CTLA-4-induced colitis in IL-10−/− mice. Mice were treated with DSS in drinking water for 1 week followed by a recovery period of another week, and administered 100 μg of anti-CTLA-4 mAb and 3-IAld as depicted in FIG. 2A. Mice were evaluated for (FIG. 6A) % weight change, (FIG. 6B) colon histology (PAS staining) and (FIG. 6C) histology score, Photographs were taken with a high-resolution microscope, ×20 magnification (scale bars, 200 μm). Data are represented as mean±SEM. H₂O, untreated mice. *P<0.05, **p<0.01.

FIGS. 7A-7B are graphical representations and photograph images showing how 3-IAld protects in IL-10−/−mice from pathology after acute treatment with anti-CTLA-4 monoclonal antibody. Mice were administered 100 μg of anti-CTLA-4 mAb and 3-IAld as described above and evaluated for (FIG. 7A) % weight change and (FIG. 7B) colon histology (PAS staining). Photographs were taken with a high-resolution microscope, ×20 magnification (scale bars, 200 μm).

FIGS. 8A-8H are graphical representations and photograph images showing how 3-IAld protects IL-10-deficient mice from pathology after chronic treatment with anti-CTLA-4 monoclonal antibody. Control C56BL/6 and IL-10-deficient mice were administered 100 μg of anti-CTLA-4 mAb and 3-IAld as depicted in the experimental schedule (FIG. 8A). Mice were evaluated for (FIG. 8B) weight change, (FIG. 8C) disease activity index, (FIG. 8D) clinical inspection, (FIG. 8E) colon and (FIG. 8F) histology (PAS staining) (in the insets, ZO-1 and BrdU staining), (FIG. 8G) calprotectin levels in gut homogenates and (FIG. 8H) cytokine and defensing gene expression in the gut. Photographs were taken with a high-resolution microscope, ×20 magnification (scale bars, 200 μm). Data are represented as mean±SEM (pooled from two experiments). H₂O, untreated mice. *P<0.05, **p<0.01.

FIGS. 9A-9B are graphical representations and photograph images showing how 3-IAld reduces lymphocytic infiltration in IL-10-deficient mice after chronic treatment with anti-CTLA-4 monoclonal antibody. Mice were treated as in legend to FIG. 8A and assessed for histological changes and lymphocytic infiltration (quantified by using the ImageJ software) by anti-CD3 staining in different organs. DAPI (4′,6-diamidino-2-phenylindole) for DNA staining.

FIGS. 10A-10D are graphical representations and photograph images showing how 3-IAld slows disease progression in 16 wk-old IL-10-deficient mice. Mice were treated with 3-IAld and 1% DSS as depicted in (FIG. 10A) and assessed for (FIG. 10B) % body weight, (FIG. 10C) histology score and (FIG. 10D) colon inflammatory histopathology and epithelial and renewal, by PAS and Ki-67 staining, respectively.

FIGS. 11A-11F are graphical representations and photograph images showing how 3-IAld-modified microbiota provides protection in DSS+anti-CTLA-4-induced colitis. C57BL/6 mice were subjected to DSS-colitis with (FIG. 11E) or without (FIGS. 11A-11D) anti-CTLA-4 treatment and transplanted with fresh fecal pellets (FMT) from control or 3-IAld-treated mice 1 day before and 2 days after colitis induction. Mice were sacrificed 7 (FIG. 11A-11D) or 14 (FIG. 11E) days after colitis induction and evaluated for (FIGS. 11A, 11E) % weight change, (FIG. 11B) gross pathology, (FIG. 11C) histology score, (FIG. 11D) colon histopathology (periodic acid-Schiff staining), (FIG. 11F) methylation/demethylation status of Foxp3 promoter in mesenteric lymph nodes. Photographs were taken with a high-resolution microscope, ×10 and ×20 magnification (scale bars, 500 and 200 μm). Data are representative of three independent experiments. Each in vivo experiment includes 3 mice per group (6-12 mice in each experiment). Data are represented as mean±SD. H₂O, untreated mice. *P<0.05, **P<0.01, ***P<0.001.

FIGS. 12A-12J are graphical representations and photograph images showing how 3-IAld does not interfere with the development of antitumor immunity. For FIGS. 12A-12E, C57BL/6 mice were subcutaneously injected with B16 tumor cells and administered 100 μg anti-CTLA-4 mAb or isotype control intraperitoneally four times at 3-day intervals up to 16 days. 3-IAld was administered intragastrically every other day. Mice were evaluated for (FIG. 12A) tumor growth, (FIG. 12B) histology (periodic acid-Schiff staining), (FIG. 12C) immunohistochemistry for CD8+ and CD4+ tumor-infiltrating lymphocytes (TIL), (FIG. 12D) number of positive TIL per high-power field, HPF and (FIG. 12E) Cxcl9 and Perforin expression. (FIGS. 12F-12J) C57BL/6 mice were orthotopically injected with LLC cells and administered 200 μg anti-PD-1 mAb or isotype control intraperitoneally five times at 3-day intervals up to 18 days. 3-IAld was administered intragastrically every other day. Mice were evaluated for (FIG. 12F) % survival, (FIG. 12G) lung weight, (FIG. 12H) lung gross pathology, (FIGS. 12I, 12J) CD4+CD25+Foxp3+ cells. Photographs were taken with a high-resolution microscope, ×40 magnification (scale bars, 100 μm). Each in vivo experiment includes three mice per group (nine mice in each experiment). Data are represented as mean±SEM. One-way analysis of variance, Bonferroni post hoc test. *P<0.05, **P<0.01, ***P<0.001.

FIGS. 13A-13E graphically represent pharmacodynamics studies including analysis of unlabelled 3-IAld detected small variations over basal endogenous levels of 3-IAld at the different time points (FIG. 13A), analysis of labelled 3-IAld (FIG. 13B), analysis of potential metabolites of unlabelled 3-IAld (FIG. 13C), levels of methylated forms of indole-3-formic acid, namely 1-Methylindole-2-carboxylic acid and Methyl indole-3-carboxylate (FIG. 13D), and the presence of indole-3-formic acid was also confirmed with labelled 3-IAld (FIG. 13E).

FIG. 14 graphically represents Cypla1 mRNA fold increase with the addition of 3-IAld or Ox-3-IAld. Human cell lines, HepG2, a liver cancer cell line, were exposed to different concentrations of either ligand Indole-3-formic acid, or Ox-3-IAld, showing induction of the AhR activation marker Cyp1A1 in a dose-dependent manner between 1 and 100 μM (FIG. 14).

FIG. 15 pictorially shows tissue pathology blindly examined on different organs after Hematoxylin and Eosin (H&E) staining.

FIG. 16 is a representation of the chemical structure of 1-methylindole-3-carboxylic acid, also known as 1-methyl-3-indolecarboxylic acid, 1-Methyl-1H-indole-3-carboxylic acid, or 1ME3CA (MF: C10H9NO2, IUPAC name: 1-Methyl-1H-indole-3-carboxylic acid).

FIGS. 17A-17F are graphical representations and photograph images showing that 1-methylindole-3-carboxylic acid protects from inflammatory pathology in DSS+anti-CTLA-4-induced colitis. C57BL/6 mice were treated with DSS in drinking water for 1 week followed by a recovery period of another week and administered 100 μg of anti-CTLA-4 mAb and 1-methylindole-3-carboxylic acid as depicted in the experimental schedule, wherein the 1-methylindole-3-carboxylic acid was administered at dosages of either 0.09, 0.18, or 0.36 mg/mouse every other day (FIG. 17A). Mice were evaluated for (FIG. 17B) weight change, (FIG. 17C) disease activity index, (FIG. 17D) clinical morbidity and rectal bleeding, (FIGS. 17E-17F) colon and ileum histology via PAS staining, epithelial barrier function (ZO-1 staining) and lymphocytic infiltration (CD3 staining) in the colon and ileum. Photographs were taken with a high-resolution microscope, ×20 magnification (scale bars, 200 μm). Each in vivo experiment includes four to six mice per group (16-24 mice in each experiment). Data are represented as mean±SEM. H₂O, untreated mice. One-way analysis of variance, Bonferroni post hoc test. ***P<0.001, ****P<0.0001.

FIG. 18 are photograph images showing that 1-methylindole-3-carboxylic acid reduces lymphocytic infiltration in anti-CTLA-4-treated mice. C57BL/6 mice were treated with DSS in drinking water for 1 week followed by a recovery period of another week and administered 100 μg of anti-CTLA-4 mAb and 1-methylindole-3-carboxylic acid as described in the experimental schedule of FIG. 17A. Mice were assessed for histological changes and lymphocytic infiltration by anti-CD3 staining in the lung. DAPI (4′,6-diamidino-2-phenylindole) was used for DNA staining.

FIG. 19 graphically represents Il1b, I110, Cyp1a1, Reg3g, and Il22 mRNA fold increase showing that the addition of 1-methylindole-3-carboxylic acid promotes an anti-inflammatory state and AhR activation in murine colitis. C57BL/6 mice, subjected to DSS+anti-CTLA-4-induced colitis and administered 1-methylindole-3-carboxylic acid as described in the experimental schedule of in FIG. 17A, were evaluated for cytokine and AhR-dependent gene expression. Data are represented as mean±SEM. H₂O, untreated mice. *P<0.05, **P<0.01, ***P<0.001, ****P<0.0001. One-way ANOVA, Bonferroni post hoc test.

FIG. 20A shows the pharmacokinetics of 3-IAld in intestine, serum, lung, liver, brain, and kidney over a 24 hours time period. FIG. 20B shows the pharmacokinetics of 1-methylindole-3-carboxylic acid in intestine, serum, lung, liver, brain, and kidney over a 24 hours time period.

FIG. 21 shows the AhR agonistic activity of 3-IAld and 1ME3CA in vitro in the reporter H1L6.1c3 cell line.

FIG. 22 shows the AhR activity of 3-IAld and 1ME3CA in vitro in A549 alveolar epithelial cells (FIG. 22A); Calu-3 bronchial epithelial cells (FIG. 22B); Caco-2 colon carcinoma cells (FIG. 22C); and HepG2 hepatic carcinoma cells (FIG. 22D). ITE and FICZ are reference AhR agonists. Assays were performed at 4 hours post-incubation.

FIG. 23 shows that MECA protects from inflammatory pathology in DSSS+ anti-CTLA-4 induced colitis in a dose-dependent manner. C57BL/6 mice were treated with DSS in drinking water for 1 week followed by a recovery period of another week and administered 100 g of anti-CTLA-4 mAb and MECA as depicted in the experimental schedule (FIG. 23A). Mice were evaluated for (FIG. 23B) weight change, (FIG. 23C) disease activity index, (FIG. 23D) clinical morbidity and rectal bleeding, (FIG. 23E) colon and ileum histology (PAS staining), ×40 magnification (scale bars, 100 m). Each in vivo experiment includes four to six mice per group (20 mice in each experiment).

FIG. 24 shows that MECA reduces lymphocytic infiltration in anti-CTLA-4-treated mice: dose-dependent activity. Lung, spleen, liver, and kidney tissue samples showed a dose-dependent ability to reduce lymphocytic infiltration in anti-CTLA-4-treated mice that had been given MECA.

FIGS. 25A and 25B shows the toxicological effects of 3-IAld and 1ME3CA on PAS-stained tissue sections on naïve C57BL/6 mice. Photographs were taken using a high-resolution Olympus DP71 microscope using a 10× objective. Scale bar 400 m. Naive, untreated mice.

### DETAILED DESCRIPTION

Some aspects of the present disclosure are directed to a method of treating a CTLA-4 checkpoint related immunodeficiency in a patient comprising administering a therapeutically effective amount of a pharmaceutical composition comprising 1H-indole-3-carboxaldehyde (3-IAld) to the patient in need thereof.

Additional aspects of the present disclosure are directed to a method of treating a CTLA-4 checkpoint related immunodeficiency in a patient comprising administering a therapeutically effective amount of a pharmaceutical composition comprising 1-methylindole-3-carboxylic acid to the patient in need thereof.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

As described herein, any concentration range, percentage range, ratio range or integer range is to be understood to include the value of any integer within the recited range and, when appropriate, fractions thereof (such as one tenth and one hundredth of an integer), unless otherwise indicated.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). In addition, “or” is used mean an open list of the components in the list. For example, “wherein X comprises A or B” means X comprises A, X comprises B, X comprises A and B, or X comprises A or B and any other components.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, a patient who is “homozygous” for a particular gene mutation has the same mutation on each allele.

As used herein, a patient who is “heterozygous” for a particular gene mutation has this mutation on one allele, and a different mutation on the other allele.

An “antibody” can include e.g. monoclonal antibody (mAb), polyclonal, multispecific (for example bispecific), recombinant, human, chimeric and humanized antibodies. Furthermore, the term “antibody” can also encompass recombinantly expressed antigen binding proteins and antigen binding synthetic peptides.

An “antigen” refers to any molecule, e.g., a peptide, that provokes an immune response or is capable of being bound by a TCR. The immune response may involve antibody production, the activation of specific immunologically-competent cells, or a combination thereof. A person of skill in the art would readily understand that any macromolecule, including virtually all proteins or peptides, can serve as an antigen. An antigen can be endogenously expressed, i.e. expressed by genomic DNA, or can be recombinantly expressed. An antigen and/or an epitope can be specific to a certain tissue, such as a cancer cell, or it can be broadly expressed. In addition, fragments of larger molecules can act as antigens. In one aspect, antigens are tumor antigens.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival of a patient, an increase in life expectancy of a patient, or amelioration of various physiological symptoms in a patient associated with the tumor. An anti-tumor effect can also refer to the prevention of the occurrence of a tumor, e.g., a vaccine.

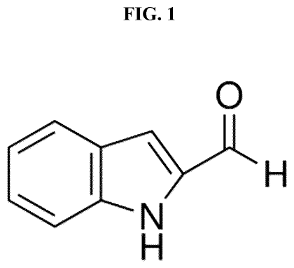

“Indole-3-aldehyde” or “3-IAld” is a metabolite derived from the microbial degradation of the amino acid tryptophan. Identification of 3-IAld is further described in US Pat. Appl. Pub. No. 2016/0206595, which is herein incorporated by reference. 3-IAld is an agonist of the Aryl Hydrocarbon Receptor (AhR), a ligand-activated transcription factor involved in a wide range of physiological activities, including the maintenance of the mucosal homeostasis at barrier organs (Stockinger B, et al., Ann. Rev. Immunol. 2014; 32:403-32.) By activating AhR in type 3 innate lymphoid cells, 3-IAld induces the production of IL-22, a key cytokine in host defense at mucosal surfaces and tissue repair, and in the regulation of microbial composition (Zelante T, et al., Immunity. 2013; 39(2):372-85; Borghi M, et al., Immunol. 2019; 10:2364.) The 3-IAld-AhR-IL-22 axis thus represents a functional signaling unit for the enhancement of barrier functions with promising activity in pathological conditions characterized by epithelial damage, mucosal alterations, and hyperinflammatory responses. The formula for indole-3-aldehyde (or indole-3-carbaldehyde, 3-IAld, MF: C9H7NO, name IUPAC: 1H-indole-3-carbaldehyde) is shown below and in FIG. 1. 3-IAld is expected to be well tolerated in humans too as it is free from any potential side effect.

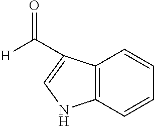 [SMILES: O=Cc1c[nH]c2ccccc12]

Endogenous tryptophan (Trp) metabolites have an important role in mammalian gut immune homeostasis. In the gastrointestinal tract, diet derived AhR ligands promote local IL-22 production (Lee et al, 2011) by innate lymphoid cells (ILCs) (Qiu et al, 2012), now referred to as group 3 TLCs (ILC3s) (Spits et al, 2013). Metabolomic analysis has revealed that gut bacteria impact host metabolism and immunity through a variety of chemically different metabolites, including amino acid metabolites (Wikoff et al, 2009). In particular, dietary lack of Trp impairs intestinal immunity in mice and alters the gut microbial community (Hashimoto et al, 2012), suggesting that mucosal homeostasis is a multifactorial phenomenon of which Trp metabolism is an important regulatory component. However, the source and nature of any such AhR ligands, any impact of microbial dysbiosis on AhR- and IL-22-driven mucosal reactivity, and whether AhR activation by microbiota-derived metabolites also occurs have all been unclear. Although the enzyme Trp 2,3-dioxygenase (Opitz et al, 2011), mainly expressed in the liver, regulates Trp concentrations after nutritional Trp uptake under normal circumstances, the high amounts of IDO1 expression at mucosal sites during immune activation (Dai et al, 2010) point to IDO1 as the dominant enzyme regulating the local amino acid nutrient levels, the size and metabolic activity of gut microbiota, and, owing to the host's own immunomodulatory activity via L-kynurenine production, mucosal immune reactivity. Thus, these data qualify IDO1 as a key molecule in dictating host-microbiota symbiotic relationships and their integration within the adaptive immunity of vertebrate hosts.

The microbiota-AhR axis might represent an important strategy pursued by coevolutive commensalism for fine tuning host mucosal reactivity contingent on Trp catabolism. To this end, the drug 3-IAld may offer a significant advantage over any other current treatment by improving host immune reactivity, epithelial barrier function and pathogen colonization. By acting as ligands of the AhR, a transcription factor that controls biodegradation of endogenous and exogenous toxins, prevents inflammatory damage and provides barrier integrity (Hubbard et al, 2015 b), indoles exert multiple functions that include bidirectional communication with the microbiome for fine tuning host immunity, tolerance and metabolism (Stockinger et al, 2014). It has been shown that indole administration to germ-free mice increased the expression of epithelial tight junction proteins and attenuated indicators of inflammatory colitis (Shimada et al, 2013). Similarly, a therapeutic efficacy in a murine model of dextran-induced colitis has been reported for indole-3-aldehyde (3-IAld) (Zelante et al, 2013).

As used herein, the term “active pharmaceutical ingredient” or “therapeutic agent” (“API”) refers to a biologically active compound.

The terms “patient” and “subject” are used interchangeably and refer to an animal including humans.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

A “CTLA-4 checkpoint related immunodeficiency” or “primary CTLA-4 checkpoint related immunodeficiency” or immunodeficiencies are disorders or diseases in a patient characterized by variable combination of enteropathy, hypo-gammaglobulinemia, recurrent respiratory infections, granulomatous lymphocytic interstitial lung disease, lymphocytic infiltration of non-lymphoid organs (intestine, lung, brain, bone marrow, kidney), autoimmune thrombocytopenia or neutropenia, autoimmune haemolytic anaemia and lymphadenopathy. CTLA-4 checkpoint related immunodeficiencies are diagnosed based on clinical symptoms, laboratory findings, and genetic testing. Patients with only one functional copy of the gene encoding CTLA-4 suffer from severe autoimmunity. These heterozygous mutations result in a new phenotype, with infiltration of nonlymphoid organs, such as the intestine, lungs, and brain, by hyperactive T cells and B cells, along with more classic signs of autoimmunity. Some clinical manifestations in CTLA-4-haploinsufficiency are reminiscent of those observed in biopsies of inflamed organs. Patients with a primary CTLA-4 checkpoint related immunodeficiency may have recurrent respiratory tract infections, hypo-gammaglobulinemia, autoimmune cytopenia, autoimmune enteropathy and granulomatous infiltrative lung disease.

Primary CTLA-4 checkpoint related immunodeficiencies are diagnosed based on clinical symptoms, laboratory findings, and genetic testing. Patients with only one functional copy of the gene encoding CTLA-4 suffer from severe autoimmunity. These heterozygous mutations result in a new phenotype, with infiltration of nonlymphoid organs, such as the intestine, lungs, and brain, by hyperactive T cells and B cells, along with more classic signs of autoimmunity. Some clinical manifestations in CTLA-4-haploinsufficiency are reminiscent of those observed in biopsies of inflamed organs in patients receiving anti-CTLA-4 therapy. Patients with a primary CTLA-4 checkpoint related immunodeficiency may have recurrent respiratory tract infections, hypo-gammaglobulinemia, autoimmune cytopenia, autoimmune enteropathy and granulomatous infiltrative lung disease.

“CTLA-4” is an essential negative regulator of T cell-mediated immune responses. The essential role of CTLA-4 in lymphocyte homeostasis and tolerance is clearly showed by ctla4 knockout mice, which develop rapidly fatal destructive multiorgan lymphocytic infiltration. Autoimmunity arises from self-reactive T and/or B lymphocytes, and underlies a wide range of conditions, from endocrine disorders to blood cytopenia. Genetic epidemiological studies have long suggested that many autoimmune conditions have an inherited component. Haploinsufficiency is defined by the occurrence of a phenotype in heterozygotes despite the lack of negative dominance of the mutant allele over its wild-type counterpart. Heterozygous CTLA-4 germline mutations impair the suppressive function of T cells and cause an immune dysregulation syndrome characterized by an activated immune system with autoimmune features and organ pathology caused by infiltrating effector T cells. So, having a single working copy of CTLA-4 is not sufficient to produce enough CTLA-4 protein for a normal immune system. These heterozygous mutations result in a new phenotype, with infiltration of intestine, lungs, and brain, by hyperactive T cells and B cells, along with more classic signs of autoimmunity.

Located contiguously on the long arm of the second chromosome are gene paralogs encoding the immunoglobulin-family co-activation receptors CD28 and CTLA-4. CD28 and CTLA-4 share the same B7 ligands yet each provides opposing proliferative signals to T cells. Whereas CD28 delivers a positive signal into T cells leading to T cell activation and effector cell differentiation, the ligation of CTLA-4 to CD80 and CD86 delivers a negative signal to T cells, limiting IL-2 production, proliferation, and survival of T cells. CTLA-4 is expressed inducibly on CD4+ Foxp3 conventional T (Tconv) cells after activation and constitutively on CD4+ Foxp3+ regulatory T (Treg) cells. The critical inhibitory function of CTLA-4 has been revealed by the rapidly fatal inflammatory phenotype of CTLA-4-null (Ctla4^{-/-}) mice, which spontaneously develop massive T cell expansion with multiorgan lymphocytic infiltration and tissue destruction (Tivol et al, 1995). The similarity of this phenotype to systemic autoimmune disease has catalyzed studies investigating the role of CTLA-4 in T cell tolerance and autoimmunity. CTLA-4 can act on Tconv and Treg cells and is a mediator of Treg cell suppressive function.

Human CTLA4 haploinsufficiency caused dysregulation of FoxP3+ regulatory T (Treg) cells, hyperactivation of effector T cells, and lymphocytic infiltration of target organs such as the intestine, lungs and brain along with more classic signs of autoimmunity. Adaptive immune responses must balance the response against foreign antigens with the need to avoid damage to self-antigens and host tissue. At one end of the spectrum, inefficient activation of the immune response results in pathology due to infections whereas overactivation may drive an autoimmune response. It might be expected that distinct genetic mutations underlie these apparently opposite outcomes, yet paradoxically it is well recognized that autoimmunity and immunodeficiency can manifest concurrently in the same individuals.

Since CTLA-4 inhibits the CD28 pathway, which plays a role in T cell help for B cell responses, deficiency in CTLA-4 might be expected to enhance CD28 function and promote humoral immunity. One possible explanation is that hyperactivation of T cells may result in infiltration and disruption of the bone marrow niche impairing B cell development. This is consistent with the disruptions in B cell lymphopoiesis in Treg-deficient mice. Alternatively, increased CD28-dependent follicular helper T (TFH) cell differentiation could result in chronic stimulation of B cells leading to exhaustion.

CTLA-4 deficiency is characterized by infiltration of immune cells into the gut, lungs, bone marrow, central nervous system, kidneys, and possibly other organs. Most people with CTLA4 deficiency experience diarrhea or intestinal disease. Enlarged lymph nodes, liver, and spleen also are common, as are respiratory infections. People with CTLA-4 deficiency often experience autoimmune problems that can affect various organs and tissues, including the blood, thyroid, skin, and joints. The disease also may slightly increase the risk of lymphoma, a type of immune-cell cancer.

The increased cancer risk, combined with the observation of viral association, leads us to assume that a defective immune surveillance of chronically virus-infected cells and the reduced elimination of oncogenic viruses leads to deregulated cell growth. Decreased CTLA-4 expression results in uncontrolled proliferation of T cells with a possible overgrowth of autoreactive clones over e.g., EBV-specific T cell clones, as known for persons with HIV infection.

It is worth nothing that autosomal dominant CTLA-4 deficiency is characterized by incomplete penetrance, as some heterozygous individuals are asymptomatic. It is well known that a delicate balance exists between self-tolerance and autoimmunity that is governed, at least in part, by quantitative variations in CTLA-4 expression. As predicted by preclinical models, the complex interplay between genetics and the environment might determine the evolution of distinct phenotypes associated with CTLA-4 deficits, or conversely, the maintenance of an asymptomatic state (Kuehn et al, 2014).

CTLA-4 checkpoint related immunodeficiencies lead to a syndrome of immune dysregulation with a broad spectrum of clinical manifestations. In many cases, onset of clinical manifestations is not observed until adulthood. Some patients suffer of severe haematological cytopenia. Patients with immune thrombocytopenic purpura are at risk of bleeding complications, those with immune haemolytic anaemia may have complications secondary to hypoxia including the risk of acute renal failure and acute vascular events. In patients with pulmonary lymphocyte infiltration, recurrent pulmonary infectious episodes may occur, sometimes aggravated by the presence of hypogammaglobulinemia and concomitant immunosuppressive therapy established to reduce lymphocyte infiltration and autoinflammation. Some patients also suffer from gastrointestinal disorders. Those with gastric atrophy are at risk of ulcers, also due to the tendency to develop recurrent Infections by Helicobacter pylori can progress to a lymphoproliferative disorder (for example a MALT lymphoma). Severe complications that can alter the quality and quantity of life are colitis, pancreatitis, and infiltration of the central nervous system. (Schubert et al, 2014; Kuehn et al, 2014).

The report by Kuehn et al. describing the consequences of decreased CTLA-4 expression bears notable similarities to, as well as distinctions from, reports of inflammatory disorders associated with anti-CTLA-4 cancer therapy. Among 540 melanoma patients receiving intermittent CTLA-4 blockade with ipilimumab, approximately 60% experienced immune-related adverse events, and 11% had severe symptoms, the most common of which were dermatologic (rash and vitiligo), gastrointestinal (enterocolitis), and endocrine (hypothyroidism and hypophysitis) (Hodi et al, 2010). Less common inflammatory events included hepatitis, uveitis, neurologic disorders, and pneumonitis (Attia et al, 2005). Although most immune-related toxicities were readily managed with immunosuppressive drugs, some were fatal. Biopsies of inflamed organs demonstrated mixed CD4+ and CD8+ T cell infiltrates. Increased serum titers of autoantibodies observed in some patients were directed against thyroid tissue, acetylcholine receptor, pituitary gland, and other targets.

A significant correlation between severe immune-related toxicities and major tumor regressions was described (Attia et al, 2005), suggesting common biological mechanisms and highlighting the precarious balance between self-tolerance and autoimmunity in malignant and normal tissues. Cancer predisposition in CTLA-4 insufficiency appears as a result of immune activation with chronic inflammation, failure to control oncogenic viruses or neoplastic cells by immunological means, plus an intrinsic T cell impairment due to the underlying genetic defect (Salavoura et al, 2008). For these reasons, patients with CTLA-4 haploinsufficiency have an increased susceptibility to malignancies, in particularly gastric cancer and lymphomas (Dhalla et al, 2011—Cunningham-Rundles et al, 1999). Lymphomas are known to occur as a result of the lymphopenia present in most patients. Gastric cancers often arise from a chronic inflammatory tissue, which is a commonly known risk factor (Compare et al, 2010). A recent study by Egg et al demonstrated an elevated risk for malignancies of 12.9% in affected CTLA4 mutation carriers. In addition, comparing the risk of cancer between the general population and affected CTLA4 mutations carriers, affected CTLA4 mutation carriers had a higher cancer rate per year (Egg et al, 2018).

As such patients with CTLA-4 checkpoint related immunodeficiencies or primary CTLA-4 checkpoint related immunodeficiencies may exhibit several different gastroenteric diagnosis, syndromes, or symptoms including but not limited to CHAI, LRBA, LATAIE, regulatory T cell defects, enteropathy disease, gut inflammation, immune-mediated colitis (IMC), gastrointestinal disorders, gastric atrophy, colitis, as well as other that may not be fully recognized at this time.

As used herein “CTLA-4 haploinsufficiency with autoimmune infiltration (CHAI)” is a CTLA-4 checkpoint related immunodeficiency disorder that is characterized by heterozygous CTLA4 mutation(s) in humans associated with a severe immunoregulatory disorder. CHAI disease is predominantly characterized by lymphoproliferation, autoimmune cytopenia, enteropathy, interstitial lung disease and recurrent infections.

As used herein “lipopolysaccharide-responsive beige-like anchor (LRBA)” is a CTLA-4 checkpoint related immunodeficiency disorder which is characterized by gene harboring genetic biallelic mutations mutation(s) in patients. As the LRBA deficiency also causes a secondary loss of CTLA-4, this recessive disease is called LRBA deficiency with autoantibodies, T-reg cell defects, autoimmune infiltration and enteropathy (LATAIE). LATAIE also generally presents with enteropathy, autoimmunity, lymphoproliferation and respiratory infections. Actually, in CHAI patients the rates of granulomas, malignancies, atopy, cutaneous disorders and neurological disorders are higher, while LATAIE patients are more commonly complicated with life-threatening infections, pneumonia, ear, nose and throat disorders, organomegaly, autoimmune enteropathy and growth failure. Although features of CHAI and LATAIE are similar, a notable difference is the typically earlier age of onset with LATAIE, where disease onset is often apparent in preschool age children, whereas CHAI presents in older children or young adults.

As used herein a “regulatory T (Treg) cell defect” is a CTLA-4 checkpoint related immunodeficiency disorder which refers to any defective immune surveillance response which may lead to chronically virus-infected cells and reduced elimination of oncogenic viruses which leads to deregulated cell growth. One example is the decreased CTLA-4 expression which results in uncontrolled proliferation of T cells with a possible overgrowth of autoreactive clones over e.g., EBV-specific T cell clones.

As used herein “enteropathy disease” is a CTLA-4 checkpoint related immunodeficiency disorder which refers to ongoing damage or irritation and swelling of the small intestine.

As used herein “gut inflammation” is a CTLA-4 checkpoint related immunodeficiency symptom which refers to inflammation of the gastrointestinal (GI) tract. Prolonged inflammation results in damage to the GI tract.

As used herein “immune-mediated colitis (IMC)” is a common immune related adverse event associated with immune checkpoint inhibitors involving abdominal pain, mucus or blood in the stools, and fever. Immune checkpoint inhibitors are immunotherapy drugs that work by blocking checkpoint proteins from binding with their partner proteins. This prevents the “off” signal from being sent, allowing the T cells to kill cancer cells. The dysregulation in T cells may also contribute to IMC, wherein the T cells attack the GI track cells and tissues.

As used herein an “autoimmune infiltration” or “infiltration” is the diffusion or accumulation (in a tissue or cells) of foreign substances in amounts excess of the normal. Infiltration may include lymphocytic infiltration of non-lymphoid organs such as the intestine such as in CHAI. Weakening of the intestinal lining may also lead to infiltration with other infiltrates in the intestine.

As used herein “gastric atrophy” is a condition marked by thinning of the inner lining of the stomach and/or intestinal wall and the loss of gland cells in the lining that release substances that help with digestion. It may be caused by infection with the bacterium H. pylori or by certain autoimmune conditions.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some embodiments, the methods of the present invention can be used to reduce the tumor size of a tumor derived from, for example, the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, breast cancer, prostate cancer, lung cancer (e.g., non-small cell lung cancer (NSCLC) or small cell lung cancer (SCLC)), Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. The particular cancer can be responsive to chemo- or radiation therapy or the cancer can be refractory. A refractory cancer refers to a cancer that is not amendable to surgical intervention, and the cancer is either initially unresponsive to chemo- or radiation therapy or the cancer becomes unresponsive over time.

It is understood that wherever aspects are described herein with the language “comprising,” otherwise analogous aspects described in terms of “consisting of” and/or “consisting essentially of” are also provided.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell, added to a cell in culture, administered to a subject, or any combination thereof. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, IL-21, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1a (MIP-1a, MIP-1a), MIP-1b (MIP-1b), gamma-induced protein 10 (IP-10), and thymus and activation regulated chemokine (TARC or CCL17).

Other examples of cytokines include, but are not limited to chemokine (C—C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), leukemia inhibitory factor (LIF), oncostatin M (OSM), CD 154, lymphotoxin (LT) beta, 4-IBB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell. In some aspects, an immune response refers to NK cell-mediated killing of a foreign cell, e.g., an allogeneic T cell therapy.

“Immunotherapy” refers to the treatment of a subject afflicted with, or at risk of contracting or suffering a recurrence of, a disease by a method comprising inducing, enhancing, suppressing or otherwise modifying the immune system or an immune response.

As used herein, the term “inactivating” or “inactivation,” e.g., in reference to a gene or protein, refers to a measure that can induce the reduced expression of the protein. In some aspects, inactivation can be achieved by a deletion or mutation of all or a part of the coding region of a gene or all or part of a non-coding region of a gene that results in decreased expression of the gene or the protein encoded by the gene. In some aspects, inactivation is achieved by deletion of the entire coding region of a gene. In some aspects, inactivation is achieved by partial deletion of a coding region of a gene. In some aspects, inactivation is achieved by deletion of one or more regulatory elements that facilitate expression of the gene. In some aspects, inactivation is achieved by a mutation in one or more regulatory elements that results in decreased expression or loss of expression of the gene. In some aspects, inactivation is achieved by mutation of one or more nucleic acid that results in the expression of a non-functional protein. In some aspects, inactivation is achieved by a missense mutation that results in the expression of a non-functional protein. In some aspects, inactivation is achieved by interference of the transcription or translation of a gene that results in the reduced expression of the protein. In some aspects, decreased expression is relative to the expression of the target gene in the cell prior to modification (e.g., deletion or mutation). In some aspects, the expression of the gene is measured prior to modification, then the cell is modified, and then the expression of the gene is measured following modification.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses by inducing apoptosis or programmed cell death in the target cell. They were termed “natural killers” because NK cells do not require activation in order to kill a target cell. T-cells play a major role in cell-mediated-immunity. T-cell receptors (TCR) expressed on the surface of T cells differentiate T cells from other lymphocyte types. The thymus, a specialized organ of the immune system, is primarily responsible for T cell maturation. There are six types of T-cells, namely: Helper T-cells (e.g. CD4+ cells); Cytotoxic T-cells (also known as TC, cytotoxic T lymphocyte, CTL, T-killer cell, cytolytic T cell, CD8⁺ T-cells or killer T cell); Memory T-cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2R.p, CXCR3, and LFA-1, and show numerous functional attributes distinctive of memory cells); (ii) central memory TCM cells express L-selectin and the CCR7, they secrete IL-2, but not IFNy or IL-4, and (iii) effector memory TEM cells, however, do not express L-selectin or CCR7 but produce effector cytokines like IFNy and IL-4); Regulatory T-cells (Tregs, suppressor T cells, or CD4+CD25+ regulatory T cells); Natural Killer T-cells (NKT); and Gamma Delta T-cells.

B-cells play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

Regulatory T cells (Tregs) are a specialized subpopulation of T cells that act to suppress immune response, thereby maintaining homeostasis and self-tolerance. It has been shown that Tregs are able to inhibit T cell proliferation and cytokine production and play a critical role in preventing autoimmunity. Different subsets with various functions of Treg cells exist. Tregs can be usually identified by flow cytometry. The most specific marker for these cells is FoxP3, which is localized intracellulary. Selected surface markers such as CD25high (high molecular density) and CD127low (low molecular density) could serve as surrogate markers to detect Tregs in a routine clinical practice. Dysregulation in Treg cell frequency or functions may lead to the development of autoimmune disease.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, polymers, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

Pharmaceutically acceptable carriers may comprise a polymer, polymers, or polymer mixes. One example polymer mixture is the Eudrgit® (Evonik Industries AG, Essen, Germany). EUDRAGIT® polymers are easy to handle and process at any scale, with multiple options available for supply as aqueous dispersions, granules, organic solutions, powders, or ready-to-use powders. EUDRAGIT® polymers are compatible with all relevant process technologies including film coating, melt, wet or dry granulation, hot melt extrusion, micro-encapsulation and spray drying. Furthermore, all of our polymers are manufactured to a consistent high-quality, with established, audited sites helping to provide global supply security. EUDRAGIT® polymers can be used individually or in combination to match virtually any target release profile including immediate, delayed, sustained, pulsatile, accelerated and zero order release. Standard options per coating layer include the use of a single EUDRAGIT® polymer, or a combination of either EUDRAGIT® polymers or with other polymers, as well as certain other oral excipients and drug substances. Evonik's proprietary AEMP® (Advanced Excipient Manufacturing Process) technology may also be leveraged to combine the functional benefits of different EUDRAGIT® polymers to create new combination polymers that can further improve functionality and create new opportunities in formulation development and drug product design. Pharmaceutically acceptable carriers may be designed for immediate, delays, or sustained release.

The term “pharmaceutically acceptable” as used herein can in particular indicate that the “pharmaceutically acceptable” compound or “pharmaceutically acceptable” composition is suitable for administration to a subject to achieve a treatment and/or prevention of a disease, of a disorder or of a condition, in particular of at least one of a CTLA-4 checkpoint related immunodeficiency.

A pharmaceutical composition of the present invention can be in solid or liquid form and can be, inter alia, in a form of one or more powder(s), one or more tablet(s), one or more fluids, in particular one or more solution(s), or one or more aerosol(s). A pharmaceutical composition of the invention can also comprise one or more further biologically active agent(s), such as for example active agent(s), e. g. 3-IAld, for use in the treatment and/or prevention of at least one of a CTLA-4 checkpoint related immunodeficiency. The administration of a pharmaceutical composition of the present invention can be for example an administration selected from the group consisting of intraperitoneal, intravenous, parenteral, intrarenal, subcutaneous, topical, intrabronchial, intrapulmonary and intranasal administration and, if desired for local treatment, intralesional administration. An enteric administration can be for example an oral administration or other means of delivering 3-IAld to the gut and GI tract. The compositions of the invention can also be administered directly to the target site, e.g., by biolistic delivery to the target site, like a specific organ afflicted with a disease, disorder or condition.

In particular, said administration can be carried out by injection and/or infusion and/or delivery, such as e.g. intravenous or intraperitoneal injection or infusion. The pharmaceutical composition can be present in the form of an injectable dosage form or a dosage form for administration by infusion, in particular in the form of an injectable dosage form for intravenous or intraperitoneal injection or an infusion dosage form for intravenous or intraperitoneal administration, or a solid dose such as delivered enterically by oral administration such as by a tablet, capsule formation, or other solid or semi-solid formulation such as a dry chewable or a aqueous based gel or soft-gel like, or gummy formulation.

A pharmaceutical composition according to the present invention can be administered to the subject at a suitable dose. The dosage regimen can be for example determined by an attending physician. As well known in the art, dosages for a patient can depend upon many factors, such as the patient's size, body surface area, age, weight, administration for prevention or treatment purposes, target indication, the particular compound to be administered, general health, and other drugs being administered concurrently. According to one embodiment, at least one antibody of the present invention.

According to one aspect, a pharmaceutical composition of the present invention can be a pharmaceutical composition which comprises 3-IAld and optionally at least one pharmaceutically acceptable carrier.

Moreover, doses of 3-IAld and optionally at least one pharmaceutically acceptable carrier of the present invention below or above the above indicated exemplary ranges can be administered, e.g. for treating and/or preventing at least one of CTLA-4 checkpoint related immunodeficiency. A pharmaceutical composition of the present invention can be formulated to be short-acting, fast-releasing, long-acting, or sustained-releasing.

Furthermore, a pharmaceutical composition of the present invention can comprise further biologically active agents, depending on the intended use of the pharmaceutical composition.

As used herein, the terms “reduced expression” and “increased expression” refer to the expression of a particular gene or protein in a cell relative to a control, e.g., the expression of a particular gene in a modified cell as compared to the expression of the gene in a wild-type (unmodified) cell. The relative expression can be based on mRNA levels and/or protein levels. Any means of measuring the level of mRNA and/or protein can be used to determine whether a gene or protein has reduced or increased expression, including but not limited to immunohistochemistry and PCR-based techniques.

As used herein, the terms “subject” and “patient” are used interchangeably and refer to either a human or a non-human, such as primates, mammals, and vertebrates. In particular aspects, the subject is a human.

The term “therapeutic benefit” or “therapeutically effective” as used herein, refers to anything that promotes or enhances the well-being of the subject with respect to the medical treatment of this condition. This includes, but is not limited to, a reduction in the frequency or severity of the signs or symptoms of a disease.

The term “effective amount” or “therapeutic dose” or “therapeutically effective dose” is a dose or concentration of a drug that produces a positive biological response. For purposes of the present disclosure an aspect of an effective amount may produce reduced symptoms of inflammation, infiltration, weight loss, irritation, diarrhea, bloody stool, mucus in the stool, and tissue and cell damage.

As used herein, the term “treating” or “treatment” of a disease or condition refers to executing a protocol, which may include administering one or more therapies to a patient, in an effort to alleviate signs or symptoms of the disease. In some aspects, a treatment decreases the rate of disease progression, ameliorates or palliates the disease state, and/or facilitates remission or improved prognosis. Alleviation can occur prior to signs or symptoms of the disease or condition appearing, as well as after their appearance. Thus, in some aspects, “treating” or “treatment” includes “preventing” or “prevention” of a disease or an undesirable condition. However, “treating” or “treatment” does not require complete alleviation of all signs and/or symptoms, does not require a cure, and specifically includes protocols that have only a marginal effect on the patient.

In various aspects, a subject in need thereof may be treated for a disease or for alleviating symptoms associated with a CTLA-4 checkpoint related immunodeficiency.

As used herein, the terms “ug” and “uM“are used interchangeably with” g” and “μM,” respectively.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. As described herein, any concentration range, percentage range, ratio range or integer range is to be understood to include the value of any integer within the recited range and, when appropriate, fractions thereof (such as one-tenth and one-hundredth of an integer), unless otherwise indicated.

Abbreviations used herein are defined throughout the present disclosure. Various aspects of the disclosure are described in further detail in the following subsections.

Various aspects described herein are described in further detail in the following subsections.

### II. Compositions of the Disclosure

Some aspects of the present disclosure are directed to 3-IAld. Other aspects of the disclosure are related to a pharmaceutical composition comprising 3-IAld and a pharmaceutically acceptable carrier.

1H-Indole-3-carboxaldehyde, also known as indole-3-aldehyde or 3-formylindole (3-IAld, MF C9H7NO, IUPAC name: 1H-indole-3-carbaldehyde), belongs to the class of organic compounds known as indoles. Indoles are compounds containing an indole moiety, which consists of pyrrole ring fused to benzene to form 2, 3-benzopyrrole (FIG. 1). 1H-Indole-3-carboxaldehyde exists as a solid, slightly soluble in water, and a very weakly acidic compound (based on its pKa). 3-IAld is a metabolite of dietary L-tryptophan which is synthesized by human gastrointestinal bacteria, particularly species of the Lactobacillus genus (Zelante et al, 2013—Lamas et al, 2016).

The 3-IAld is an agonist of the Aryl hydrocarbon receptor (AhR), a ligand-activated transcription factor involved in a wide range of physiological activities, including the maintenance of the mucosal homeostasis at barrier organs (Zelante et al, 2013—Zhang et al, 2016—Stockinger et al, 2014).

AhR is a ligand-dependent basic helix-loop-helix transcription factor that is highly conserved evolutionarily and is expressed in the majority of immune cell types and human tissues (Stockinger et al, 2014). Traditionally considered for its ability to metabolize harmful toxicants via activation of the cytochrome P450 drug-metabolizing enzymes, AhR is increasingly being recognized for its multiplicity of functions, including developmental biology and cross-talk with the microbiome for regulation of host immunity, tolerance and metabolism. In particular, the ability of AhR to engage Th17 cells for antimicrobial activity, induce IL-22 production for epithelial cell repair and protection, and activate regulatory T cells for control of inflammation, makes the intestinal and respiratory barriers very sensitive to AhR activity and activation (Esser et al, 2015). Therefore, AhR ligands, such as 3-IAlD, are promising compounds for pharmaceutical drug discovery, to treat inflammatory pathology at mucosal surfaces. Several studies have highlighted the capacity of AhR to respond to indoles and indolyl metabolites, thus positioning AhR as a candidate indole receptor (Hubbard et al, 2015a). Indoles represent a wide group of gut bacteria-derived compounds produced from tryptophan, which exert significant biological effects, and may contribute to the etiology of cardiovascular, metabolic, and psychiatric diseases (Konopelski et al, 2018). However, most of the research in the field is limited to experimental studies, likely accounted for by the context- and ligand-dependent activity of AhR (Safe et al, 2020). Therefore, the activation of AhR by 3-IAld holds great therapeutic potential, provided that a ligand, such as 3-IAld, with an optimal efficacy/safety profile be precisely delivered to the target organs through suitable biopharmaceutical formulations. This appears to be the case of 3-IAld that formulated for intestine release via enteric microparticles improved inflammatory histopathology and barrier function as revealed by the production of IL-22, increased expression of the tight junction zonula occludens (ZO) 1, proliferation of intestinal Lrg5+ cells (leucine-rich repeat-containing G protein-coupled receptor 5, a stem cell marker in intestinal crypts (Kumar et al, 2014), reduced intestinal leakage and expression of Nfil3 (Yu et al, 2014), a transcription factor that directs the development of innate lymphoid cells 3 known to maintain epithelia barrier integrity (Puccetti et al, 2021).

Some aspects of the present disclosure are directed to 1-methylindole-3-carboxylic acid. Other aspects of the disclosure are related to a pharmaceutical composition comprising 1-methylindole-3-carboxylic acid and a pharmaceutically acceptable carrier.

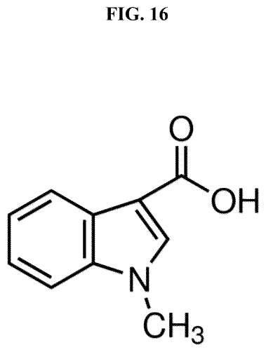

1-methylindole-3-carboxylic acid, also known as 1-methyl-3-indolecarboxylic acid or 1-Methyl-1H-indole-3-carboxylic acid (MECA) (1ME3CA) (MF: C10H9NO2, IUPAC name: 1-Methyl-1H-indole-3-carboxylic acid), belongs to the class of organic compounds known as methylindoles. 1-methylindole-3-carboxylic acid contains a methylindole moiety, which consists of a 1-methylpyrrole ring fused to benzene to form 1-methylindole (FIG. 16).

### III. Methods of Treatment

Some aspects of the present disclosure are directed to methods of treating a disease or condition in a subject in need thereof comprising administering to the subject a composition disclosed herein. In some aspects, the method comprises administering 3-IAld alone or with a pharmaceutically acceptable carrier. In one aspect, the present invention relates to improved methods of preventing and/or treating inflammation and other symptoms associated with patients having CTLA-4 checkpoint related immunodeficiency using indole-3-aldehyde (3-IAld). In another aspect, the invention relates to methods of preventing and/or treating inflammation, increasing gut health, and GI tract lining health, reducing cell infiltrate of the gut lining, and other GI, gastric, and enterical symptoms associated with patients having CTLA-4 checkpoint related immunodeficiency using an AhR ligand with biosimilar activity. In some aspects, the AhR ligand is indole-3-acetaldehyde (IAAld), indole-3-acetic acid (IAA, Indole Acetic Acid), or indole-3-lactic acid (ILA, Indole Lactic Acid).

In some aspects, the method comprises administering 1-methylindole-3-carboxylic acid alone or with a pharmaceutically acceptable carrier. In one aspect, the present invention relates to improved methods of preventing and/or treating inflammation and other symptoms associated with patients having CTLA-4 checkpoint related immunodeficiency using 1-methylindole-3-carboxylic acid. In another aspect, the invention relates to methods of preventing and/or treating inflammation, increasing gut health, and GI tract lining health, reducing cell infiltrate of the gut lining, and other GI, gastric, and enterical symptoms associated with patients having CTLA-4 checkpoint related immunodeficiency using an AhR ligand with biosimilar activity. In some aspects, the AhR ligand is 1-methylindole-3-carboxylic acid.

In some aspects, the disease or condition comprises a CTLA-4 checkpoint related immunodeficiency characterized by variable combination of enteropathy, hypo-gammaglobulinemia, recurrent respiratory infections, granulomatous lymphocytic interstitial lung disease, lymphocytic infiltration of non-lymphoid organs (intestine, lung, brain, bone marrow, kidney), autoimmune thrombocytopenia or neutropenia, autoimmune haemolytic anaemia and lymphadenopathy. CTLA-4 checkpoint related immunodeficiencies are diagnosed based on clinical symptoms, laboratory findings, and genetic testing. Patients with only one functional copy of the gene encoding CTLA-4 suffer from severe autoimmunity. These heterozygous mutations result in a new phenotype, with infiltration of nonlymphoid organs, such as the intestine, lungs, and brain, by hyperactive T cells and B cells, along with more classic signs of autoimmunity. Some clinical manifestations in CTLA-4-haploinsufficiency are reminiscent of those observed in biopsies of inflamed organs. Patients with a primary CTLA-4 checkpoint related immunodeficiency may have recurrent respiratory tract infections, hypo-gammaglobulinemia, autoimmune cytopenia, autoimmune enteropathy and granulomatous infiltrative lung disease.

One example of a CTLA-4 checkpoint related immunodeficiency is CTLA-4 haploinsufficiency with autoimmune infiltration (CHAI)” is a CTLA-4 checkpoint related immunodeficiency disorder that is characterized by heterozygous CTLA4 mutation(s) in humans associated with a severe immunoregulatory disorder. Another example of CTLA-4 checkpoint related immunodeficiency is lipopolysaccharide-responsive beige-like anchor (LRBA), a disorder which is characterized by gene harboring genetic biallelic mutations mutation(s) in patients. As the LRBA deficiency also causes a secondary loss of CTLA-4, this recessive disease is called LRBA deficiency with autoantibodies, T-reg cell defects, autoimmune infiltration and enteropathy (LATAIE).

Descriptions of asymptomatic adults with heterozygous CTLA4 deficiencies (Kuehn et al, 2014—Schubert et al, 2014), and widely varying ages of onset among symptomatic individuals, imply that additional interacting factors are required for surmounting an autoimmune threshold. These factors might include other genetic or epigenetic events and environmental influences (microbial or other). Exposure of some frequently affected organs (skin and gut) to the microbiome has suggested that this environmental factor might contribute to generating autoimmunity in patients receiving the CTLA4 blockade agent ipilimumab. Interestingly, CTLA4-mice die within 2 weeks of age even when they are rederived into germ-free environments, suggesting the importance of self-antigens in driving the inflammatory phenotype (Tivol et al, 1995).

Microbial-derived indoles are very attractive postbiotics as they have been shown to augment healthspan across a broad range of evolutionarily diverse species from different phyla (Descamps et al, 2019). The gastrointestinal tract harbors numerous species with the capacity to synthesize indole and indole-containing compounds that, by affecting host immune reactivity, epithelial barrier function and pathogen colonization, act as a possible link to microbiota dysbiosis (Roager et al, 2018). By acting as ligands of AhR, a transcription factor that controls biodegradation of endogenous and exogenous toxins, prevents inflammatory damage and provides barrier integrity (Hubbard et al, 2015 b). Indoles exert multiple functions that include bidirectional communication with the microbiome for fine tuning host immunity, tolerance and metabolism (Stockinger, 2014). It has been shown that indole administration to germ-free mice increased the expression of epithelial tight junction proteins and attenuated indicators of inflammatory colitis (Shimada et al, 2013). Similarly, a therapeutic efficacy in a murine model of dextran-induced colitis has been reported for 3-IAld (Zelante et al, 2013).

By activating innate lymphoid cells type 3 to produce IL-22, 3-IAld enhanced the barrier integrity and the production of antimicrobial peptides in murine models of colitis, gastrointestinal and vaginal candidiasis (Zelante et al, 2013; Borghi et al, 2019; Puccetti et al, 2021). It has also been shown that treatment with 3-IAld limited gut epithelial damage, reduced transepithelial bacterial translocation, and decreased inflammatory cytokine production in a murine model graft-versus-host disease (GvHD), a systemic inflammatory state initiated by donor T cells that leads to colitis. 3-IAld treatment also led to recipient-strain-specific tolerance of engrafted T cells. Transcriptional profiling and gene ontology analysis indicated that 3-IAld administration upregulated genes associated with the type I interferon response known to protect against radiation-induced intestinal damage (Swimm et al, 2018). Thus, 3-IAld by acting through different downstream effector pathways may limit intestinal inflammation and damage and may provide a therapeutic option for patients at risk for inflammatory damage of the gastrointestinal tract. This raises the possibility of developing therapeutics based on microbiota-derived indole or its derivatives to promote epithelial barrier function in humans.

Considering that CTLA-4 checkpoint related immunodeficiencies share similarity with mechanisms underlying the effects of anti-CTLA-4 therapy (Bakacs et al, 2015), this may anticipate the therapeutic potential of 3-IAld not only in CTLA-4 checkpoint related immunodeficiencies but also in preventing the adverse immune events associated with cancer treatment with immune check point inhibitors, including colitis and enteritis (Karamchandani et al, 2018; Marin-Acevedo et al, 2018). Despite the great success of cancer immunotherapy using immune checkpoint inhibitors, their therapeutic benefits are limited by either various resistance mechanisms (Schoenfeld et al, 2020) or the associated toxic effects, including frequent gastrointestinal, endocrine, and dermatologic toxicities and fatal neurotoxicity and cardiotoxicity (Choi et al, 2020). Thus, novel therapeutic strategies that provides manageable side effects to existing immunotherapy would enhance and expand their therapeutic efficacy and application. Studies have shown that the effectiveness of immunotherapy against different tumors requires the presence of commensal bacteria (Iida et al, 2013). Clinical studies have corroborated these findings with compelling evidence that microbial richness and diversity is associated with a durable response to immunotherapy with gut microbiota signatures predicting toxicity associated with combined CTLA-4 and PD-1 blockade (Andrews et al, 2021).

Another example of a CTLA-4 checkpoint related immunodeficiency is a regulatory T (Treg) cell defect which refers to any defective immune surveillance response which may lead to chronically virus-infected cells and reduced elimination of oncogenic viruses which leads to deregulated cell growth. One example is the decreased CTLA-4 expression which results in uncontrolled proliferation of T cells with a possible overgrowth of autoreactive clones over e.g., EBV-specific T cell clones.

Another example of a CTLA-4 checkpoint related immunodeficiency is enteropathy disease which refers to ongoing damage or irritation and swelling of the small intestine. Another example CTLA-4 checkpoint related immunodeficiency is gut inflammation which refers to inflammation of the gastrointestinal (GI) tract. Prolonged inflammation results in damage to the GI tract.

Another example of a CTLA-4 checkpoint related immunodeficiency is immune-mediated colitis (IMC), a common immune related adverse event associated with immune checkpoint inhibitors involving abdominal pain, mucus or blood in the stools, and fever. Immune checkpoint inhibitors are immunotherapy drugs that work by blocking checkpoint proteins from binding with their partner proteins. This prevents the “off” signal from being sent, allowing the T cells to kill cancer cells. The dysregulation in T cells may also contribute to IMC, wherein the T cells attack the GI track cells and tissues.

The therapeutic composition, e.g. 3-IAld, can be administered at a dose based on the bodyweight or mass of the individual to whom the therapeutic composition is administered. A body weight adjusted dose of the therapeutic composition can be administered from about 1 mg/kg to about 25 mg/kg. A body weight adjusted dose of the therapeutic composition can be administered from about 3 mg/kg to about 25 mg/kg, from about 10 mg/kg to about 25 mg/kg, from about 15 mg/kg to about 20 mg/kg, or from about 15 mg/kg to about 18 mg/kg. The doses of 3-IAld and/or 3-IAld pharmaceutical compositions may be delivered in immediate release, delayed release, or continuous release formulations.

The therapeutic composition, e.g. 1-methylindole-3-carboxylic acid, can be administered at a dose based on the bodyweight or mass of the individual to whom the therapeutic composition is administered. A body weight adjusted dose of the therapeutic composition can be administered from about 1 mg/kg to about 25 mg/kg. A body weight adjusted dose of the therapeutic composition can be administered from about 3 mg/kg to about 25 mg/kg, from about 10 mg/kg to about 25 mg/kg, from about 15 mg/kg to about 20 mg/kg, or from about 15 mg/kg to about 18 mg/kg. The doses of 1-methylindole-3-carboxylic acid and/or 1-methylindole-3-carboxylic acid pharmaceutical compositions may be delivered in immediate release, delayed release, or continuous release formulations.

### IV. Pharmaceutical Compositions

The compound of the invention can be incorporated into pharmaceutical compositions suitable for administration to a subject. Such compositions typically contain the active compound and a pharmaceutically acceptable carrier. As used herein the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds, such as those described above, can also be incorporated into the compositions.

A pharmaceutical composition of the invention is formulated to be compatible with its intended route of administration. Examples of routes of administration include parenteral, e.g., intravenous, intradermal, subcutaneous, oral (e.g., swallowing), transdermal (topical), transmucosal, and rectal administration. In one aspect, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The pharmaceutical compositions can be included in a container, pack, or dispenser together with instructions for administration.

### Exemplary Aspects Provided Herein

In one aspect (Aspect 1; A1), provided herein is a method of treating a CTLA-4 checkpoint related immunodeficiency in a patient comprising administering a therapeutically effective amount of a pharmaceutical composition comprising 1H-indole-3-carboxaldehyde (3-IAld) to the patient in need thereof.

In one aspect of A1, i.e., A2, the CTLA-4 checkpoint related immunodeficiency is selected from the group consisting of: CTLA-4 haploinsufficiency with autoimmune infiltration (CHAI), lipopolysaccharide-responsive beige-like anchor (LRBA) deficiency (LATAIE), a regulatory T (Treg) cell defect, autoimmune infiltration, enteropathy disease, gut inflammation, immune-mediated colitis, gastrointestinal disorder, and gastric atrophy.

In one aspect of A1 or A2, i.e., A3, the CTLA-4 checkpoint related immunodeficiency is immune-mediated colitis.

In one aspect of any one of A1 to A3, i.e., A4, the pharmaceutical composition comprises a pharmaceutically acceptable carrier.

In one aspect of A4, i.e., A5, the pharmaceutically acceptable carrier is at least one polymer.

In one aspect of A4, i.e., A6, the pharmaceutically acceptable carrier is a set of polymers.

In one aspect of A6, i.e., A7, the set of polymers are Eudragit® polymers.

In one aspect of any one of A1 to A7, i.e., A8, the pharmaceutical composition is formulated for enteric delivery.

In one aspect of any one of A1 to A8, i.e., A9, the pharmaceutical composition is orally administered.

In one aspect of any one of A1 to A9, i.e., A10, the pharmaceutical composition is in a form selected from a capsule, tablet, gel tablet, gel capsule, gel, liquid, and gummy.

In one aspect of any one of A10, i.e., A11, the pharmaceutical composition is in a tablet or capsule form.

In one aspect of any one of A1 to A11, i.e., A12, the pharmaceutical composition is administered at an interval of every other day (q.o.d).

In one aspect of any one of A1 to A12, i.e., A13, the pharmaceutical composition is administered at a 3-IAld dosage of at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, least about 16 mg/kg, at least about 17 mg/kg, or least about 18 mg/kg.

In one aspect of A13, i.e., A14, the 3-IAld dosage is about 18 mg/kg.

In one aspect, i.e., A15, provided herein is a method of treating a CTLA-4 checkpoint related immunodeficiency in a patient comprising administering a therapeutically effective amount of a pharmaceutical composition comprising 1-methylindole-3-carboxylic acid to the patient in need thereof.

In one aspect of A15, i.e., A16, the CTLA-4 checkpoint related immunodeficiency is selected from the group consisting of: CTLA-4 haploinsufficiency with autoimmune infiltration (CHAI), lipopolysaccharide-responsive beige-like anchor (LRBA) deficiency (LATAIE), a regulatory T (Treg) cell defect, autoimmune infiltration, enteropathy disease, gut inflammation, immune-mediated colitis, gastrointestinal disorder, and gastric atrophy.

In one aspect of A15 or A16, i.e., A17, the CTLA-4 checkpoint related immunodeficiency is immune-mediated colitis.

In one aspect of any one of A15 to A17, i.e., A18, the pharmaceutical composition comprises a pharmaceutically acceptable carrier.

In one aspect of A18, i.e., A19, the pharmaceutically acceptable carrier is at least one polymer.

In one aspect of A18, i.e., A20, the pharmaceutically acceptable carrier is a set of polymers.

In one aspect of A20, i.e., A21, the set of polymers are Eudragit® polymers.

In one aspect of any one of A15 to A21, i.e., A22, the pharmaceutical composition is formulated for enteric delivery.

In one aspect of any one of A15 to A22, i.e., A23, the pharmaceutical composition is orally administered.

In one aspect of any one of A15 to A23, i.e., A24, the pharmaceutical composition is in a form selected from a capsule, tablet, gel tablet, gel capsule, gel, liquid, and gummy.

In one aspect of A24, i.e., A25, the pharmaceutical composition is in a tablet or capsule form.

In one aspect of any one of A15 to A25, i.e., A26, the pharmaceutical composition is administered at an interval of every other day (q.o.d).

In one aspect of any one of A15 to A23, i.e., A27, the pharmaceutical composition is administered at a 1-methylindole-3-carboxylic acid dosage of at least about 2 mg/kg, at least 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, least about 16 mg/kg, at least about 17 mg/kg, or least about 18 mg/kg.

In one aspect of any one of A15 to A23, i.e., A28, the pharmaceutical composition is administered at a 1-methylindole-3-carboxylic acid dosage of about 2.25 mg/kg.

The present invention is further illustrated by the following examples which should not be construed as further limiting. The contents of all references cited throughout this application are expressly incorporated herein by reference.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (TRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986)); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

### 3-IAld in Dextran Sulfate Sodium (DSS)+Anti-CTLA-4-Induced Murine Colitis Models Prevents Gut Inflammatory Pathology

Considering that CTLA-4 checkpoint related immunodeficiencies share similarity with mechanisms underlying the effects of anti-CTLA-4 therapy (Bakacs et al, 2015), it was hypothesized that there may be therapeutic potential of 3-IAld not only in CTLA-4 checkpoint related immunodeficiencies but also in preventing the adverse immune events associated with cancer treatment with immune check point inhibitors, including colitis and enteritis, e.g. enteropathy disease or gastric enteritis (Karamchandani et al, 2018; Marin-Acevedo et al, 2018). Pathology inflicted by CTLA4 blockade in a Mouse Murine model of colitis, e.g. the C57BL/6 mice, (Scott et al, 2020) is known to mimic the pathogenic effects of inherited human CTLA-4 haploinsufficiency in the gut (Bakacs et al, 2015).

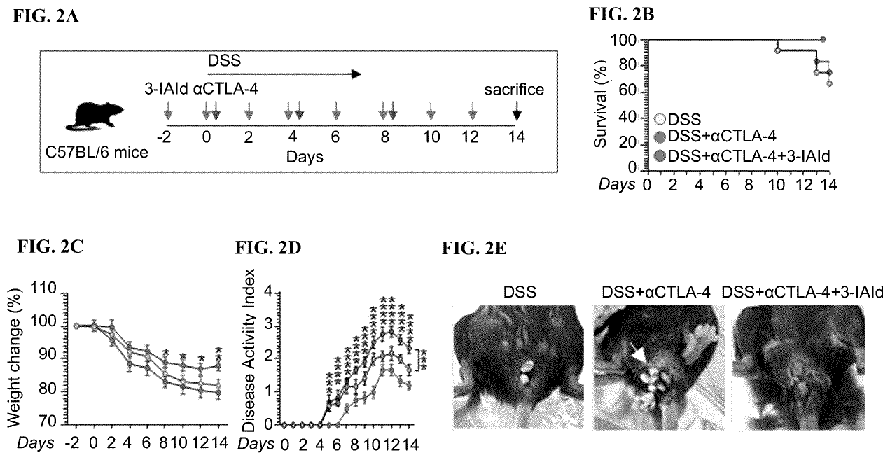

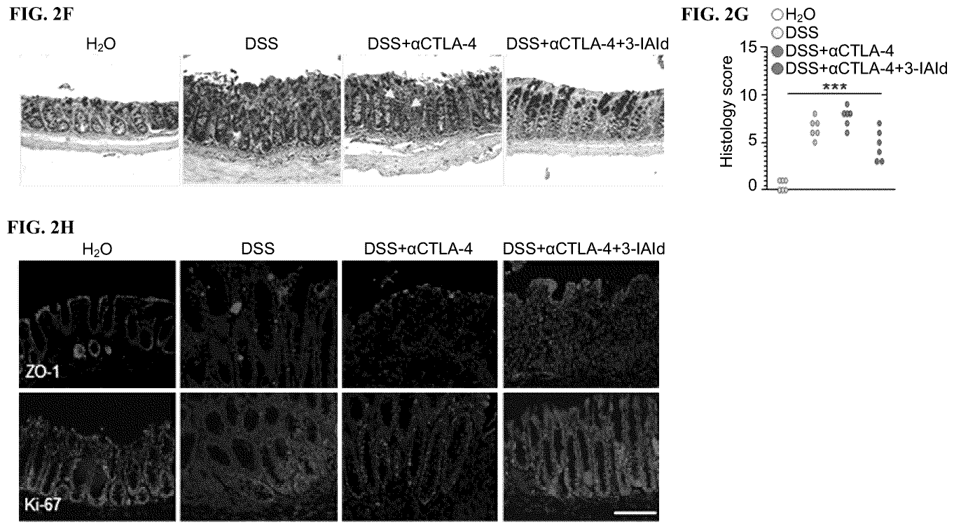

In trials, C57BL/6 mice received dextran sulfate sodium (DSS) (3%) in their drinking water and 100 μg anti-CTLA-4 monoclonal antibody (mAb) or isotype control antibody intraperitoneally, at days 0, 4 and 8 following the DSS administration as indicated in FIG. 2A. DSS is a water soluble, negatively charged sulfated polysaccharide with a highly variable molecular weight ranging from 5 to 1400 kDa. The sulfated polysaccharide does not directly induce intestinal inflammation, but rather acts as a direct chemical toxin to colonic epithelium resulting in epithelial cell injury. Eudragit-formulated 3-IAld (Puccetti et al, 2018) was given at 18 mg/kg 3-IAld intragastrically every other day, as illustrated in FIG. 2A. Animals were monitored daily for appearance of diarrhea, fecal blood, loss of body weight and survival. A week after DSS treatment (14 days after initiation of treatment), at the time at which the model recapitulates the human disease (Manicassamy et al, 2014), surviving mice were sacrificed, and the colon was excised and evaluated for macroscopic damage and local immune parameters. 3-IAld treatment increased survival (FIG. 2B) and body weight (FIG. 2C) and decreased the disease activity index (FIG. 1D). Surviving mice showed improved gross pathology (FIG. 2E) and recovery of: i) normal architecture structure of the colon (FIGS. 2F and 2G), ii) epithelial barrier function, as revealed by the expression of ZO-1 (FIG. 2H) and iii) epithelial cell proliferation and renewal, as revealed by Ki-67 staining (FIG. 2H). The results indicate 3-IAld has the ability to prevent gut inflammatory pathology inflicted by CTLA4 blockade in a murine model of colitis.

3-IAld Also Promoted the Expression of Lgr5, Nfil3 and Muc1, a Cell Surface Mucin that Functions as a Barrier to Infection and as Regulator of Inflammation.

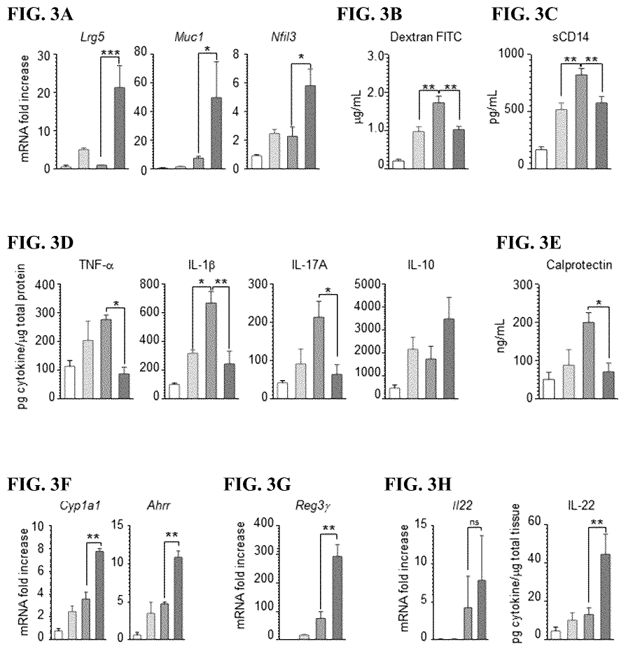

After confirming the functional recovery of the C57BL/6 mice presented in FIG. 2, it was found that 3-IAld also promoted the expression of Lgr5, Nfil3 and Muc1, a cell surface mucin that functions as a barrier to infection and as regulator of inflammation (Dhar et al, 2019) (FIG. 3A). Accordingly, the passage of dextran-FITC across the intestinal barrier was reduced (FIG. 3B), and the levels of soluble CD14, a marker of gut permeability were also decreased (FIG. 3C). These changes were paralleled by a switch towards an anti-inflammatory profile with reduced levels of TNF-α, IL-10 and IL-17A, and increased amounts of IL-10 (FIG. 3D). Consistent with the anti-inflammatory profile, the levels of calprotectin were also reduced (FIG. 3E). Considering that 3-IAld is defective in mice with colitis (Alexeev et al, 2018), these results indicate that 3-IAld supplementation may protect against DSS+anti-CTLA-4-induced colitis by maintaining epithelial barrier integrity and dampening the inflammatory response. This activity is consistent with its AhR-agonistic activity (Zelante et al, 2013—Puccetti et al. 2021) as shown by the induced expression of the AhR-dependent genes Cyp1a1 and Ahrr (FIG. 3F), of the antimicrobial peptide Reg37 (FIG. 3F) and, importantly of IL-22 (FIG. 3G, both gene and protein expression), a critical mediator of mucosal functional activity in response to 3-IAld (Renga et al. 2022).

### Example 2

### 3-IAld in Immune-Mediated Colitis Models

The activity of 3-IAld was assessed in immune-mediated models of colitis that, at variance with the chemically-induced colitis models, are considered to best recapitulate pathomechanisms of the immune-dependent colitis such as that occurring in patients with CTLA-4 haploinsufficiency (Constant et al, 2022) or treated with checkpoint inhibitors (Westdorp et al, 2021). Because deletion of CTLA-4 in mice leads to massive lymphoproliferation and fatal multiorgan tissue destruction, causing an early lethality (Tivol et al, 1995), alternative reference models of immune-mediated colitis (Kiesler et al, 2015) were used.

Humanized immunodeficient NSG mice were injected with human peripheral blood mononuclear cells from healthy donors and treated with anti-CTLA-4 monoclonal antibody. NOD.Cg-Prkdcscid Il2rgtm1Wjl/SzJ (NSG) mice, 8-10 weeks old, were injected intraperitoneally with human peripheral blood mononuclear cells (107) freshly isolated from healthy donors and treated intraperitoneally with 100 μg of anti-CTLA-4 mAb or human IgG as antibody control at days 0, 4, 8, 12 and 16.

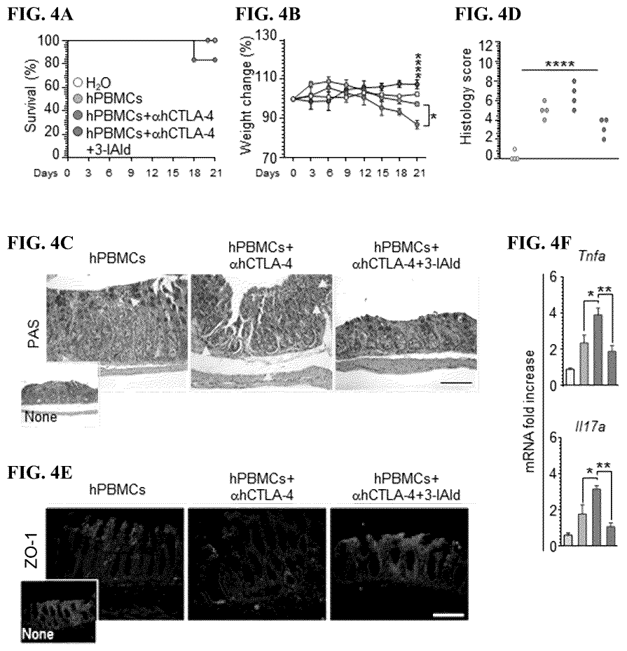

3-IAld (18 mg/kg) was administered intragastrically every other day starting the day of cell infusion. Mice were monitored daily for overall mortality and body weight loss and sacrificed on day 21. The results showed that treatment with 3-IAld increased survival (FIG. 4A), reduced weight loss (FIG. 4B), improved inflammatory histopathology (FIG. 4C) and histology score (FIG. 4D), restored barrier function, as revealed by the expression of ZO-1 (FIG. 4E) and decreased inflammatory cytokine gene expression (FIG. 4F).

3-IAld limits progression of immune-mediated colitis in RAG1-deficient mice

Immunodeficient Rag1^{-/-} mice reconstituted with naïve CD4⁺ T cells and treated with anti-CTLA-4 monoclonal antibody. Rag1−/− mice, mice homozygous for the RagltmlMom mutation produce no mature T cells or B cells. Their phenotype can be described as a “non-leaky” immune deficiency. Rag1−/− 8-10 weeks old mice were injected intraperitoneally with 4×10⁵ naïve CD4⁺ T cells purified from spleens of C57BL/6 mice and treated with anti-CTLA-4 mAb or control IgG the day after T-cell reconstitution and then at days 0, 4, 8, 12, and 16.

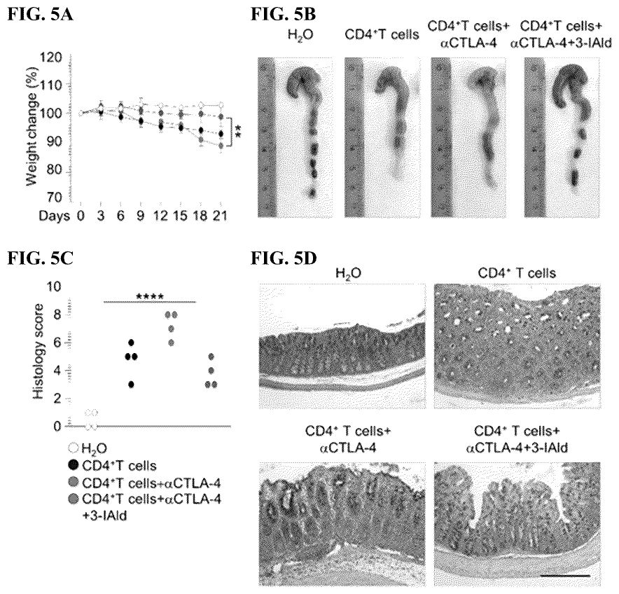

3-IAld (18 mg/kg) was administered intragastrically every other day starting the day of cell infusion. Mice were monitored daily for overall mortality and body weight loss and sacrificed on day 21. Like what observed in NSG mice, treatment with 3-IAld reduced weight loss (FIG. 5A) and improved both gross pathology (FIG. 5B) and colon histopathology (FIGS. 5C and 5D).

IL-10-Deficient (Il10^{-/-}) Mice Acutely or Chronically Treated with Anti-CTLA-4 Monoclonal Antibody, with and without DSS.

The spontaneous onset of gut inflammation after weaning, the pathological changes first seen in 3-week-old mice, has made IL-10-deficient mice (Il10^{-/-}) an excellent model to mirror the pathogenesis of immune-mediated colitis (Keubler et al, 2015) characterized by discontinuous and transmural inflammatory lesions, cell infiltrates, mainly lymphocytes, into the lamina propria and submucosa, epithelial hyperplasia, mucin depletion, ulcers, and thickening of the intestinal wall (Berg et al, 1996; Gomes-Santos et al, 2012). By 10 weeks of age, Il10^{-/-} mice developed a mild colitis the severity of which reached a plateau at 16 weeks of age (Gomes-Santos et al, 2012). We have assessed the activity of 3-IAld in Il10^{-/-} mice of different ages, i.e., 8-10 wk- or 16 wk-old, after acute or chronic treatment with anti-CTLA-4 mAb, with and without 1% DSS. The models and results are described below.

DSS+Anti-CTLA-4-Induced Colitis in 8-10 wk-Old Il^{-/-} Mice.

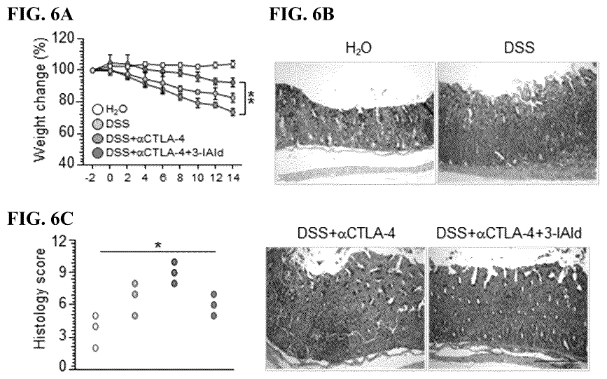

Il10^{-/-} Mice received DSS (1%) in their drinking water and 100 μg anti-CTLA-4 monoclonal antibody intraperitoneally at days 0, 4 and 8 following the DSS administration. 3-IAld-Eudragit was given (18 mg/kg 3-IAld) intragastrically every other day throughout the experiment, starting 2 days before treatment. Animals were monitored daily for body weight and inflammatory pathology in the colon until 14 days after DSS administration. The results showed the loss of body weight (FIG. 6A) and an increased epithelial thickening (FIGS. 6B and C) in 8-10 week-old IL-10-deficient mice; both signatures were exacerbated after DSS+anti-CTLA-4 treatment, being the loss of body weight further increased and the colon pathology worsened with signs of epithelia destruction and transmural infiltrates. 3-IAld treatment prevented the loss of body weight (FIG. 6A) and decreased inflammatory pathology and disease activity index (FIGS. 6B and C).

### Acute Anti-CTLA-4 Treatment in 8-10 Week-Old in Il10^{-/-} Mice

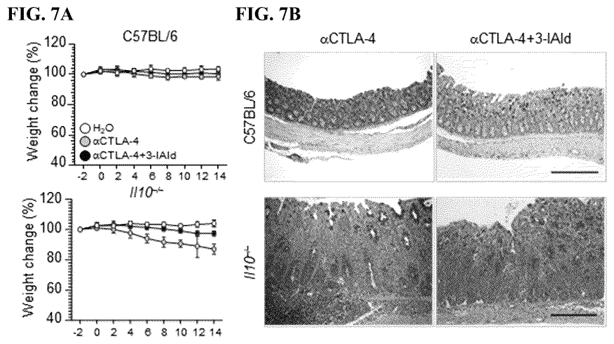

Il10^{-/-} mice were treated with anti-CTLA-4 monoclonal antibody and 3-IAld alone, as above (FIG. 6), without DSS. Animals were monitored daily for body weight and inflammatory pathology in the colon and sacrificed at 14 days after the first anti-CTLA-4 monoclonal antibody administration. The results showed that treatment with anti-CTLA-4 alone decreased the body weight (FIG. 7A) and promoted epithelial thickening associated with a massive infiltrate in the submucosa in 8-10 wk-old IL-10-deficient mice, as opposed to wild-type mice (FIG. 7B). 3-IAld treatment prevented body weight loss (FIG. 7A) and ameliorated inflammatory pathology, particularly by reducing the massive cellular infiltrate (FIG. 7B).

Chronic Anti-CTLA-4 Treatment in 16 wk-Old in Il10^{-/-} Mice

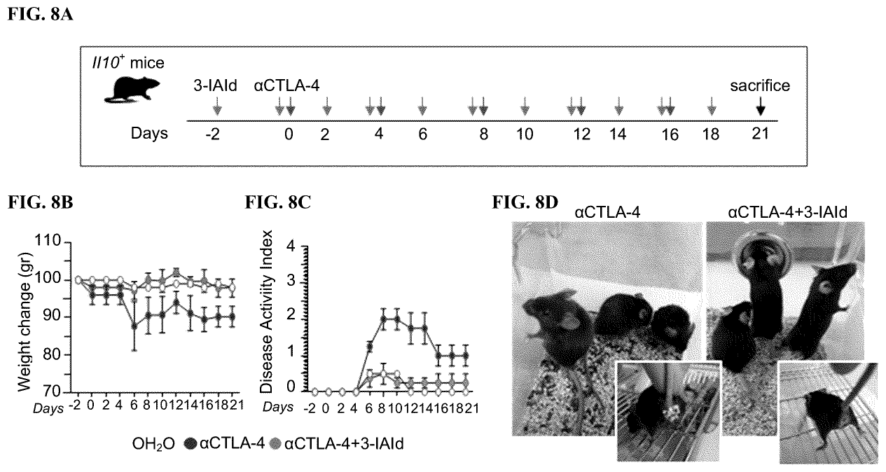

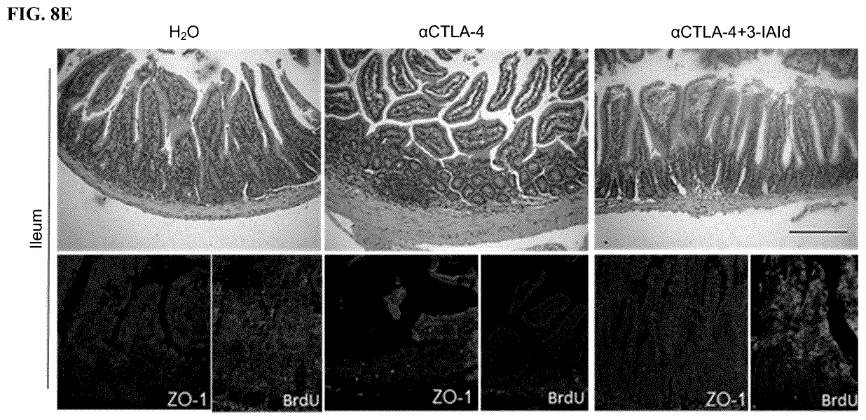

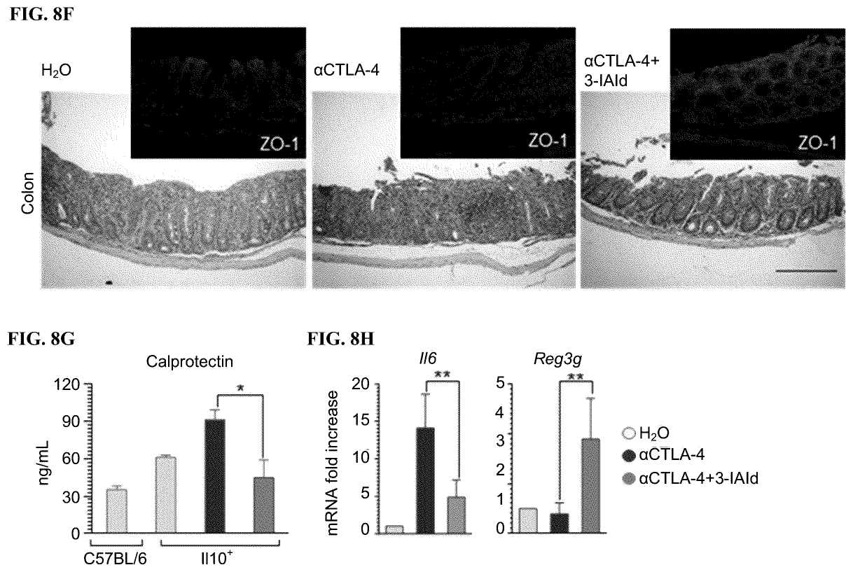

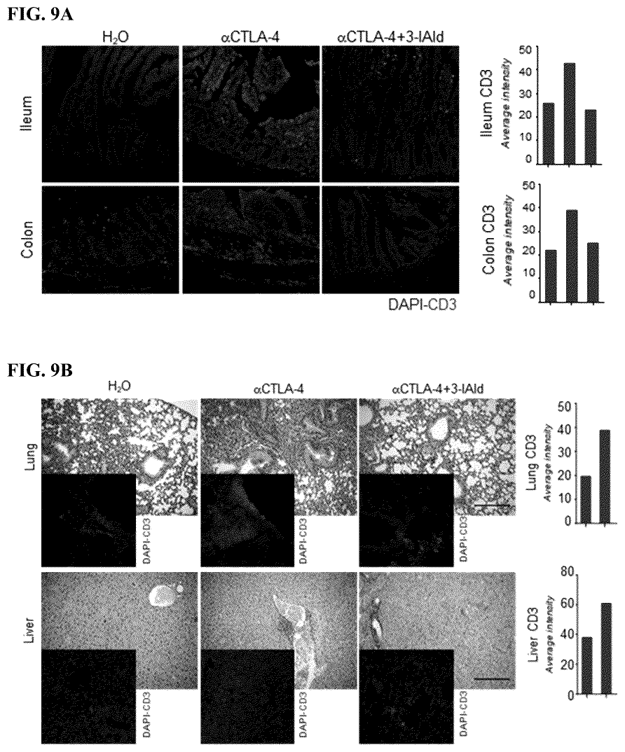

Il10^{-/-} mice were treated with 100 μg anti-CTLA-4 monoclonal antibody and 3-IAld alone, as indicated in FIG. 8A, without DSS. Animals were monitored daily for body weight, disease activity index, clinical inspection, inflammatory pathology in the ileum and colon, epithelial barrier function and immunological parameters of inflammation. The results showed that long treatment with anti-CTLA-4 alone decreased the body weight (FIG. 8B), the disease activity index (FIG. 8C), increased both clinical (FIG. 8D) and tissue pathology in the ileum and colon (FIGS. 8E and 8F), promoted epithelial damage (as revealed by the decreased expression of ZO-1) and dysfunction (as revealed by staining with bromodeoxyuridine (BrdU), a thymidine analogue that incorporates DNA of dividing cells during the S-phase of the cell cycle) and increased the levels of inflammatory calprotectin (FIG. 8G) and IL-6 (FIG. 8H), while decreasing the antimicrobial peptide Reg37 expression (FIG. 8H). Treatment with 3-IAld was effective in antagonizing the cytotoxic effects induced by CTLA-4 blockade. Consistent with the extensive T cells infiltration in different organs observed in CTLA-4 haploinsufficiency (Kuehn et al, 2014; Schubert et al, 2014; Schwab et al, 2018), we found an extensive CD3⁺Tcell infiltration in the ileum and colon (FIG. 9A), lung and liver (FIG. 9B) of mice upon anti-CTLA-4 treatment that was significantly reduced by the concomitant administration of 3-IAld as quantified in FIG. 9. No lymphocytic infiltration or immunopathology were detected in organs, such as the spleens and kidneys (data not shown).

3-IAld Treatment Slows Disease Progression in 16 wk-Old IL-10-Deficient Mice

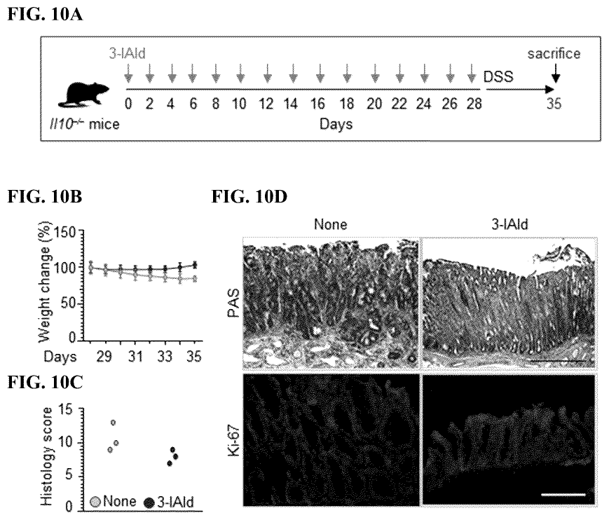

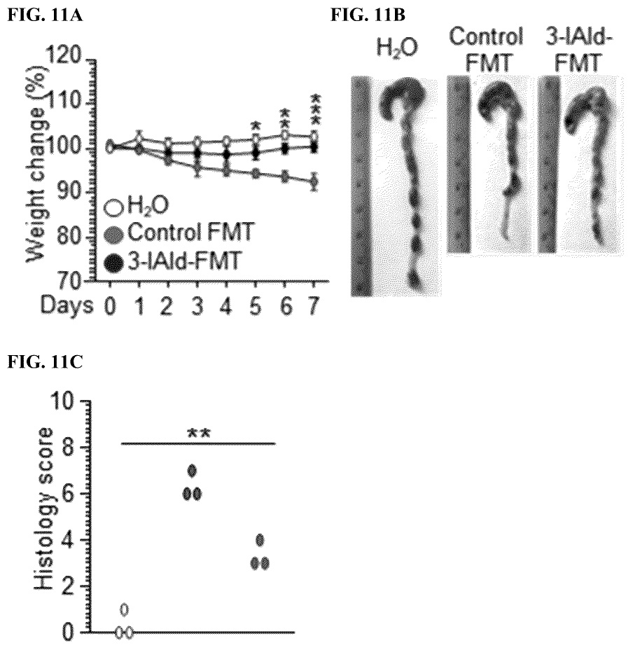

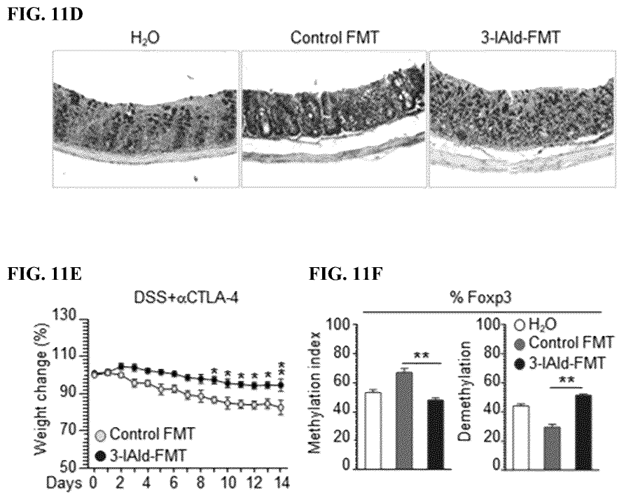

Sixteen week-old IL-10-deficient mice (Il10^{-/-}) were treated with 3-IAld every other day for 4 weeks, as illustrated in FIG. 10A. As a mimic of disease exacerbation, mice received 1% DSS after 3-IAld treatment and subsequently evaluated for weight change, intestinal pathology and epithelial integrity. The results showed that treatment with 3-IAld was apparently able to slow down the loss of body weight (FIG. 10B) and colon inflammatory histopathology (FIG. 10D) and to maintain epithelial integrity and renewal (FIG. 10D).

### Example 3

### 3-IAld Promotes Beneficial Microbiota

Given the role of the AhR/IL-22 axis in maintaining a balanced microbiota, 3-IAld was evaluated for its effect on fecal microbial composition. Fecal microbiota transplantation was used to evaluate the activity of 3-IAld-modified microbiota in colitis. Feces were collected from untreated or 3-IAld-treated mice for 6 days and transplanted (FMT) into recipient mice at the onset of colitis. At variance with transfer of feces from untreated mice, the transplantation of feces from 3-IAld-treated mice prevented weight loss (FIG. 11A), and ameliorated colon gross pathology and histopathology (FIGS. 11B-11D) in DSS-treated mice. Similar results were obtained in DSS+anti-CTLA-4-treated mice in which FMT from 3-IAld-treated mice also prevented weight loss (FIG. 11E) and induced IL-10-producing regulatory T cells (Treg), as revealed by the reversal of DNA hypermethylation of Foxp3 promoter (FIG. 11F). Overall, these results indicate that the beneficial activity of 3-IAld may occur through different pathways that include the increase intestinal barrier via the AhR/IL-22 axis, the modification of the composition and function of the microbiota and the control of inflammation via Treg cells.

### Example 4

3-IAld does not Interfere with the Development of Antitumor Immunity

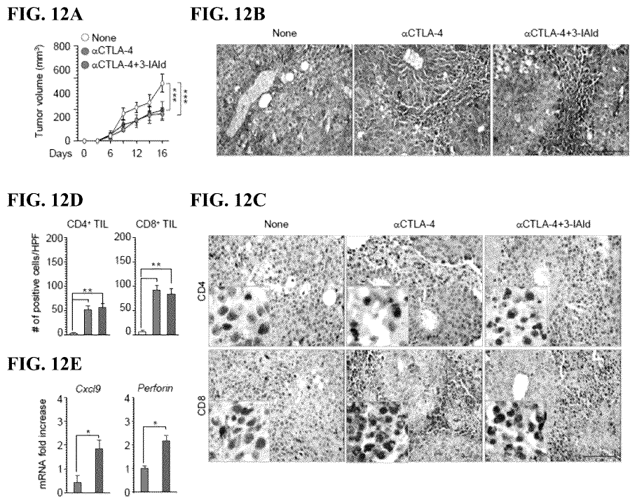

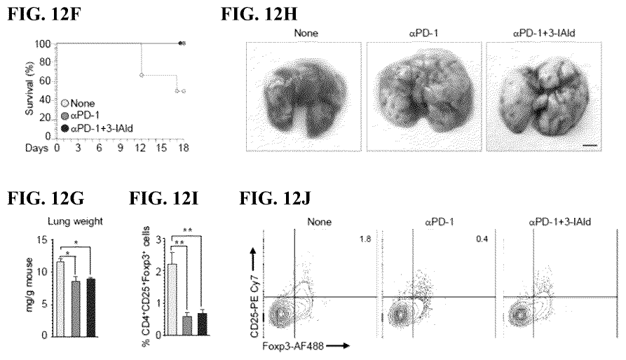

The potential application of 3-IAld requires that its immunoregulatory activity would not impinge the tumor immunosurveillance. To this purpose, the effect of 3-IAld was assessed in the anti-CTLA-4-responsive B16 melanoma model (Renga et al, 2022). 3-IAld neither modified tumor growth nor interfered with the therapeutic efficacy of anti-CTLA-4 antibody (FIGS. 12A-12B) and did not affect recruitment of CD4⁺ and CD8⁺ tumour-infiltrating lymphocytes (FIGS. 12C-12D), consistent with the increased expression of leukocyte-recruiting chemokine Cxcl9 and effector perforin (FIG. 12E). Likewise, 3-IAld did not interfere with the therapeutic efficacy of anti-PD-1 antibody in a model of Lewis lung carcinoma (LLC). Indeed, 3-IAld did not prevent the ability of anti-PD-1 antibody to increase survival (FIG. 12F), decrease tumour growth (FIG. 12G), improve gross pathology (FIG. 12H) and reduce the recruitment of Foxp3⁺CD25⁺ Treg cells in the lung (FIGS. 12I-12J). Thus, the beneficial activity of 3-IAld does not interfere with tumor immunosurveillance.

This study is a proof-of-concept demonstration of the therapeutic potential of a bacterial metabolite, such as 3-IAld, as a biologic capable to alleviate anti-CTLA-4-induced intestinal toxicity without interfering with tumor surveillance.

This suggests that 3-IAld is a unique molecule capable of breaking the dynamic feed-forward loops of the inflammation in the gut whereby the tissue destructive effects of infiltrating lymphocytes in condition of CTLA-4 deficiency may lead to microbial dysbiosis further promoting inflammation and pathology. By dually acting on both the host and the microbe sides, 3-IAld is particularly suited to break this pathogenic vicious circle and to quash inflammation.

### Example 5

Considering that inherited human CTLA-4 haploinsufficiency shares similarity with mechanisms underlying the effects of anti-CTLA-4 therapy (Bakacs et al, 2015), a relevant model to prove the efficacy of 3-IAld is represented by a murine model of immune checkpoint inhibitors-induced colitis upon concomitant treatment with anti-CTLA-4 and Dextran sulfate sodium (DSS) (Wang et al, 2018—Wang et al, 2019; Perez Riuz et al, 2019). To further support the medical plausibility of our findings, the activity of 3-IAld was assessed in immune-mediated models of colitis that, at variance with the chemically-induced colitis models, are considered to best recapitulate pathomechanisms of the immune-dependent colitis such as that occurring in patients with CTLA-4 haploinsufficiency (Constant et al, 2022) or treated with checkpoint inhibitors (Westdorp et al, 2021).

### Pharmacology

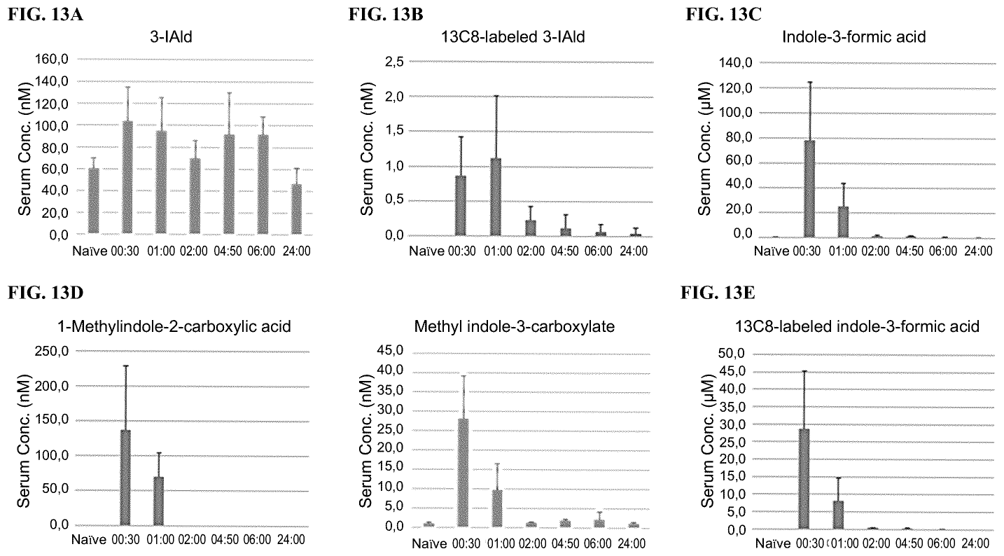

Primary pharmacodynamics studies have been performed. The mode of action of 3-IAld occurs upon binding to AhR, both in vitro and in vivo. In vitro, 3-IAld induced luciferase activity in the H1L1.1c2 cell line containing a stably-transfected AhR-responsive firefly luciferase at the dose range 0.1-100 mM (Zelante et al, 2013). In vivo, intragastric administration of 3-IAld induced IL-22 production in an AhR-dependent manner in a mouse model of mucosal candidiasis and in DSS-induced colitis (Zelante et al, 2013). Preliminary pharmacokinetics data have been obtained by administration of Eudragit-formulated 3-IAld in the composition 90% 3-IAld/10% 13C8-labeled 3-IAld and targeted/untargeted analysis by mass spectrometry in serum at different times (30 minutes (min), 1 hour (h), 2 h, 4.5h, 6 h, 24h). Analysis of unlabelled 3-IAld detected small variations over basal endogenous levels of 3-IAld at the different time points are shown in FIG. 13A. However, analysis of labelled 3-IAld revealed a peak at 30 minutes to 1 hour after administration, sharply reduced levels at 2 h while trace amounts were detected at later time points (FIG. 13B). These data demonstrate a rapid metabolic conversion of 3-IAld. Further analysis of potential metabolites of unlabelled 3-IAld revealed a peak level of the oxidated form of 3-IAld (indole-3-formic acid or Ox-3-IAld) at 30 minutes with a mean of 80 μM, rapidly diminishing at 20 μM after 1 hour and trace amounts detectable at later time points (FIG. 13C). Lower levels of methylated forms of indole-3-formic acid, namely 1-Methylindole-2-carboxylic acid and Methyl indole-3-carboxylate, were also detected with a peak at 30 minutes of about 150 and 25 nM, respectively, and reduced levels at 1 hour, thus following the same kinetics of indole-3-formic acid (FIG. 13D). The presence of indole-3-formic acid was also confirmed with labelled 3-IAld (FIG. 13E), showing a similar kinetics and a peak level of about 25 μM at 30 min.

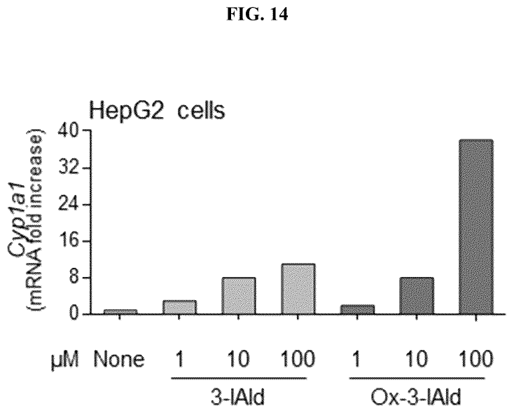

Further in vitro experiments have shown that indole-3-formic acid is pharmacologically active, thus potentially contributing to the pharmacodynamics of 3-IAld. To prove this, we exposed the human cell lines, HepG2, a liver cancer cell line, to different concentrations of either ligand Indole-3-formic acid, or Ox-3-IAld. Indole-3-formic acid, and/or Ox-3-IAld, was able to induce the AhR activation marker Cyp1A1 in a dose-dependent manner between 1 and 100 μM as shown in FIG. 14.

### Toxicology

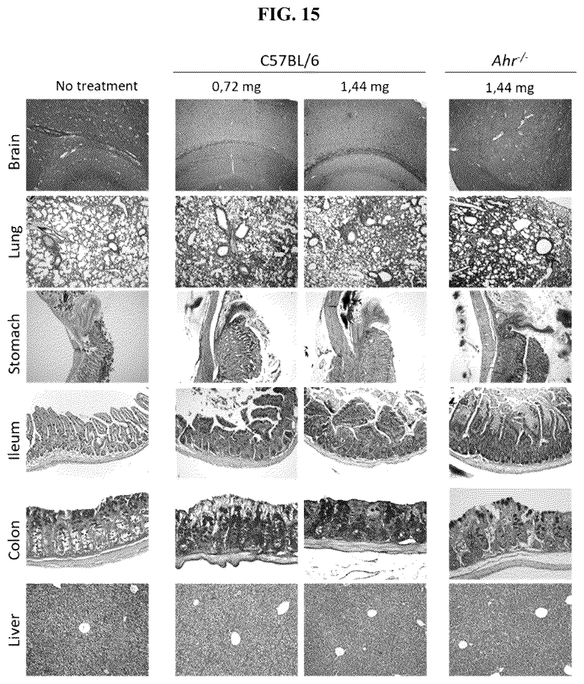

Eudragit-formulated 3-IAld (Puccetti et al, 2018) was given intragastrically every other day, at the doses indicated for 3 weeks to naive C57BL/6 and AhR−/− mice. At the end of treatment, mice were sacrificed, and tissue pathology blindly examined on different organs after Hematoxylin and Eosin (H&E) staining. The results as shown in FIG. 15 show the absence of visible tissue pathology in all the organs examined at the two different concentrations.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

While various specific aspects have been illustrated and described, the above specification is not restrictive. It will be appreciated that various changes can be made without departing from the spirit and scope of the invention(s). Many variations will become apparent to those skilled in the art upon review of this specification.

### Example 6

### 1-Methylindole-3-Carboxylic Acid for Use in Preventing Intestinal Pathology Upon CTLA-4 Blockade or Insufficiency

As presented in Example 5, a rapid metabolic conversion of 3-IAld occurs following administration. Specifically, the oxidated form of 3-IAld (indole-3-formic acid), and methylated forms of indole-3-formic acid, were detected following administration of labelled 3-IAld. For example, 1-Methylindole-3-carboxylic acid was detected with the same kinetics of indole-3-formic acid, with peak levels at 30 min (FIGS. 13C and 13D). Few studies have been performed assessing the biological activities of methylindoles and methoxyindoles as aryl hydrocarbon receptor (AhR) activators. Preliminary data obtained in vitro in cell lines indicated that 1-methylindole-3-carboxylic acid activated AhR-dependent genes in a dose-dependent manner (data not shown). Given with the ability of 3-IAld to induce AhR-dependent genes and to protect the mucosal barrier from damage, it was hypothesized that 1-methylindole-3-carboxylic acid may have similar therapeutic potential to prevent gut inflammatory pathology inflicted by CTLA-4 blockade in a murine model of colitis, known to mimic the pathogenic effects of inherited human CTLA-4 haploinsufficiency in the gut (Bakacs et al, 2015).

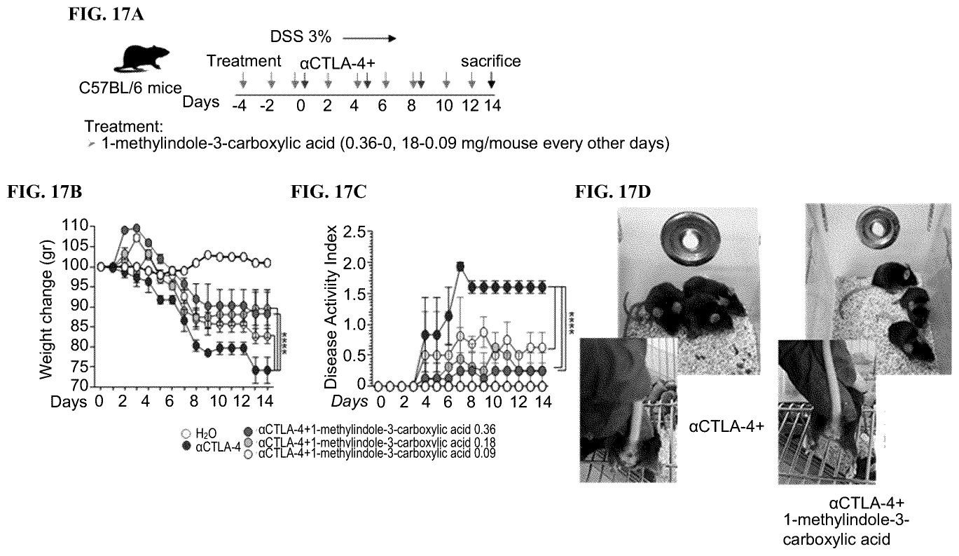

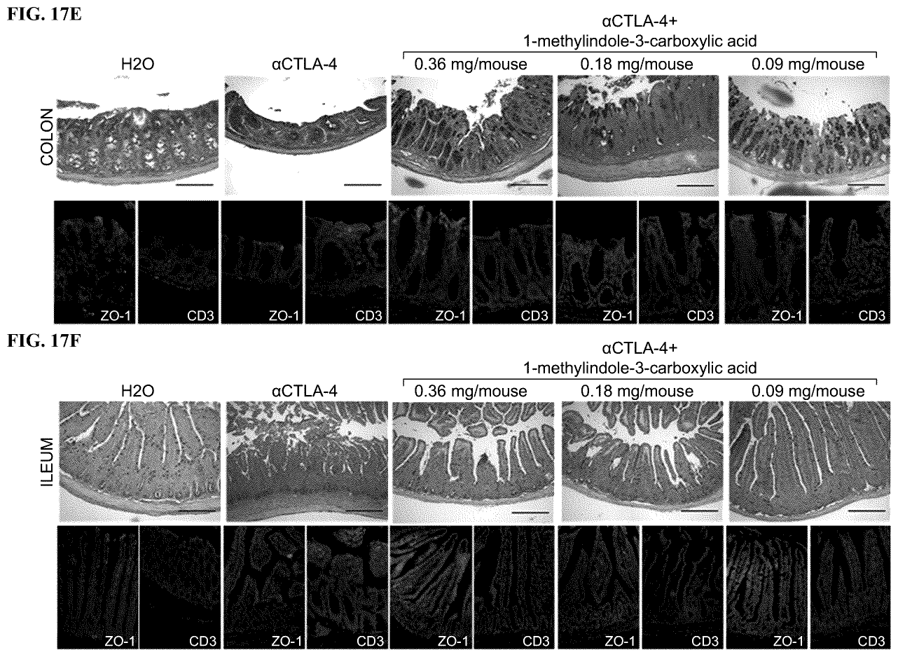

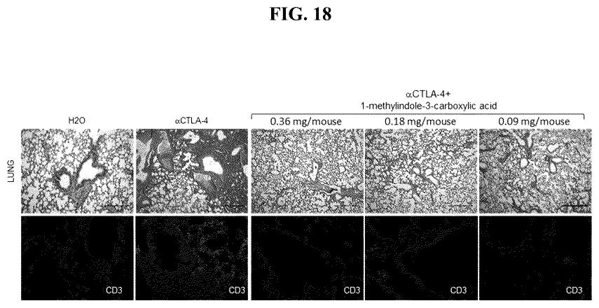

In trials, C57BL/6 mice received dextran sulfate sodium (DSS) (3%) in their drinking water and 100 μg anti-CTLA-4 monoclonal antibody (mAb) or isotype control antibody intraperitoneally, at days 0, 4 and 8 following the DSS administration as indicated in FIG. 17A. Eudragit-formulated 1-methylindole-3-carboxylic acid was given intragastrically every other day, at scaling doses of 0.09, 0.18, or 0.36 mg/mouse (FIG. 17A). Animals were monitored daily for the appearance of diarrhea, fecal blood and loss of body weight. A week after DSS treatment (14 days after initiation of treatment), at the time at which the murine model recapitulates the human disease (Manicassamy et al, 2014), surviving mice were sacrificed and the colon was excised and evaluated for macroscopic damage and local immune parameters. Mice treated with 1-methylindole-3-carboxylic acid had significant attenuation of body weight loss (FIG. 17B) and decreased disease activity index (FIG. 17C), and were protected from clinical morbidity and rectal bleeding (FIG. 17D). Positive effects were observed at each administered dose of 1-methylindole-3-carboxylic acid, including the 0.09 mg/mouse dose. Mice treated with 1-methylindole-3-carboxylic acid showed recovery of: i) normal architectural structure of the colon and ileum (FIGS. 17E-17F) and ii) maintenance of epithelial barrier function, as revealed by the expression of ZO-1 (FIGS. 17E-17F). CTLA-4 haploinsufficiency typically presents with extensive CD3+T cell infiltration across different organs (Kuehn et al, 2014—Schubert et al, 2014—Schwab et al, 2018). The extensive CD3+T cell infiltration in the ileum and colon of mice following anti-CTLA-4 treatment was significantly reduced by the concomitant administration of 1-methylindole-3-carboxylic acid (FIGS. 17E-17F). 1-methylindole-3-carboxylic acid administration was also effective in reducing CD3+T cell infiltration in the lung (FIG. 18). These results indicate that 1-methylindole-3-carboxylic acid, a metabolite of 3-IAld, has the ability to prevent gut inflammatory pathology inflicted by CTLA4 blockade in a murine model of colitis similar to treatment with 3-IAld as in FIG. 2.

### 1-Methylindole-3-Carboxylic Acid is a Potent Activator of AhR-Dependent Genes

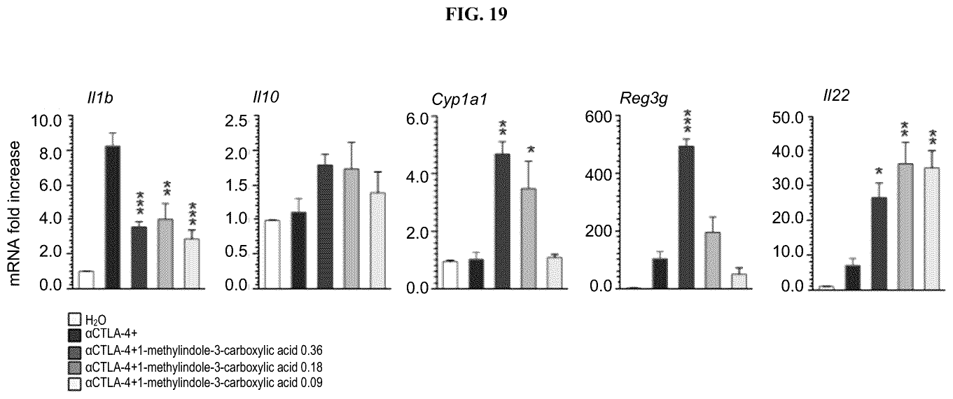

After confirming the functional recovery of the C57BL/6 mice present in FIG. 17, it was found that 1-methylindole-3-carboxylic acid also promoted an anti-inflammatory profile with reduced levels of Il1b expression and increased levels of Il10 expression in the colon (FIG. 19). These results indicate that 1-methylindole-3-carboxylic supplementation may protect against DSS+anti-CTLA-4-induced colitis by maintaining epithelial barrier integrity and dampening the inflammatory response. Consistent with its AhR-agonistic activity in vitro (data not shown), 1-methylindole-3-carboxylic induced the expression of the AhR-dependent genes Cyp1a1, the antimicrobial peptide Reg3g, and 1122 (FIG. 19), a critical mediator of the AhR-dependent mucosal protective activity (Stockinger et al, 2021). These results point to the potent activity of 1-methylindole-3-carboxylic acid in counteracting the intestinal morbidity associated with CTLA-4 deficiency upon CLTA-4 blockade, at least in part by activating the protective AhR/IL-22-dependent pathway.

### Example 7

### Pharmacology of 3-IAld/1ME3CA

### Pharmacokinetics

A single-dose pharmacokinetic (PK) study was performed on healthy C57BL/6 mice to compare 1ME3CA and 3-IAld at 0.36 mg/topo dose. Untreated naïve mice were used as the control. Four animals were sacrificed at 0.5, 2, 6, and 24 hours. Blood, obtained by cardiac puncture and collected into EDTA-containing tubes, and organs (brain, lung, intestine, liver and kidney) were assessed. All samples were stored at −80° C. until use. To evaluate 1ME3CA and 3-IAld for functional activity, the expression of AhR-downstream genes were concomitantly evaluated in these mice.

TABLE 1
Pharmacokinetics

| | 1-methylindole- | |
|---|---|---|
| | 3-carboxylic acid | 3-IAld |
| | 0.36 mg/mouse | 0.36 mg/mouse |
| Mice | (TØ) | (TØ) |
| 4 C57BL/6 | − | − |
| 20 C57BL/6 | − | + |
| 20 C57BL/6 | + | − |

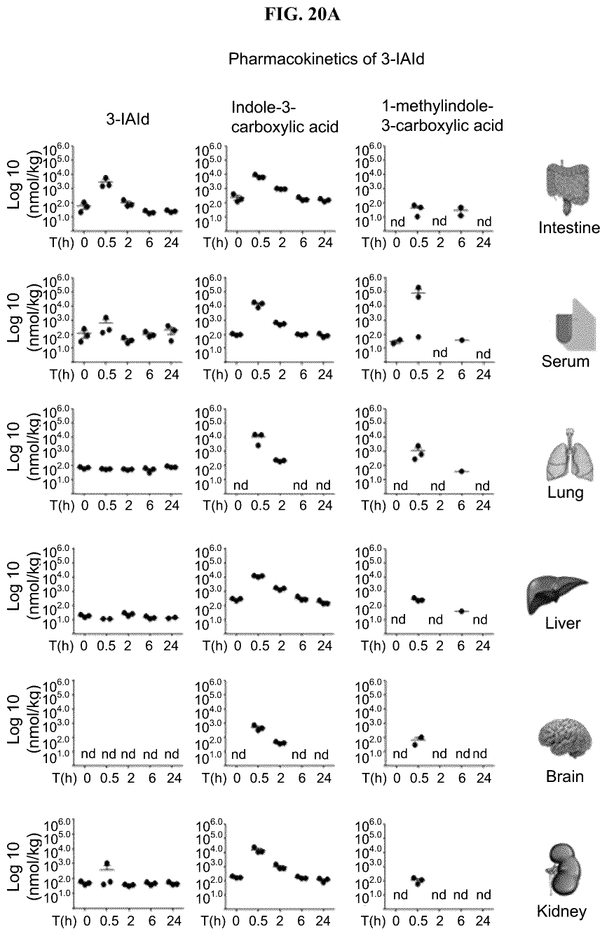

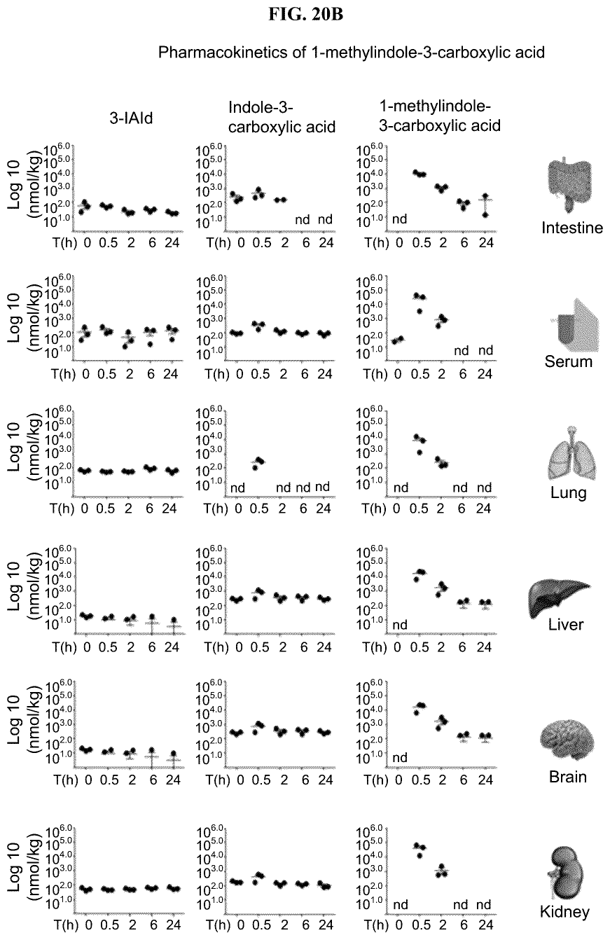

Pharmacokinetic data were obtained after a single oral administration of 18 mg/Kg of 1ME3CA or 3-IAld, both formulated in enteric microparticles, in C57BL/6 mice and targeted analysis by mass spectrometry in the intestine, serum, lung, liver, brain and kidney at different times (30′, 2 hours, 6 hours and 24 hours). As shown in FIG. 20A, 3-IAld rapidly disappeared (after 2 hours) in most tissues, terminal half-lives ranging from 3 to 5 hours, depending on tissues, as reported (Puccetti et al, Int J Pharm. 2021, 602:120610). Analysis of potential metabolites of 3-IAld revealed the presence of indole-3-carboxylic acid in serum and other tissues as a main metabolite, and to a lower extent of 1ME3CA, with a tmax of 30 minutes observed in every tissue tested, including the brain, and a rapid clearance from all the organs investigated. Compared to 3-IAld, the concentrations of 1ME3CA were basal in the intestine, serum, and kidney, while were significantly higher, between 103 to 105 nmol/kg, in the lung, liver, and the brain. Such observations suggest the formation of 1ME3CA as a secondary metabolite of 3IAld and/or indole-3-carboxylic acid. Being tmax the 3-IAld rapidly disappeared (after 2 hours) in most tissues, terminal half-lives ranging from 3 same for all compounds, an extremely rapid biotransformation of 3-IAld as well as indole-3-carboxylic acid can be theorized.

The above considerations was confirmed by looking at the analysis of 1ME3CA PK after enteric administration (FIG. 20B). Indeed, 3-IAld and indole-3-carboxylic acid concentrations were basal in all organs, which confirms the role of 1ME3CA as 3-IAld and/or indole-3-carboxylic acid metabolite. The tmax values (30 minutes) were identical and Cmax values very similar in all organs and serum. Even the profiles were similar, even though 1ME3CA was undetectable already at 6 hours in serum, lung, and kidneys. Moreover, the levels of 1ME3CA were still higher than control (at least in some organs, intestine, liver and brain) 24 hours after the administration at the time at which 1ME3CA had disappeared in mice administered with 3-IAld. Systemic levels of 1ME3CA after its oral administration seem more sustained and, as expected, significantly higher than those observed after the oral administration of 3-IAld.

### Pharmacodynamics—In Vitro Activity

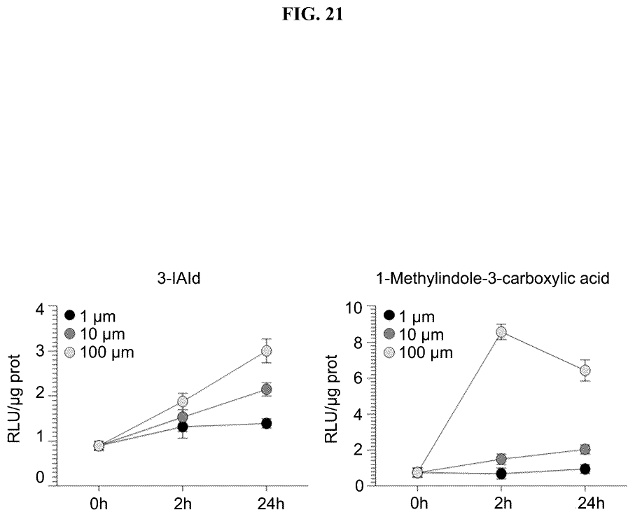

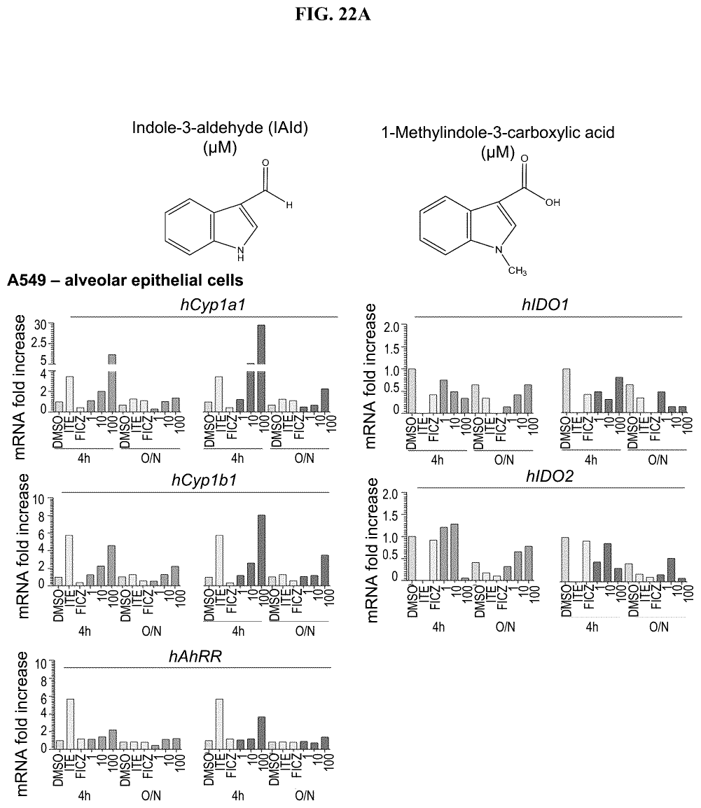

3-IAld has been shown to induce luciferase activity in the H1L1.1c2 cell line containing a stably-transfected AhR-responsive firefly luciferase at the dose range 0.1-100 μM (Zelante et al. Immunity. 2013; 39:372-85). The ability of 1ME3CA was comparatively assessed here in mouse hepatoma cells (H1L6.1c3), kindly provided by Allison K. Ehrlich (Meyer Hall, University of California, Davis, United States), containing the stably integrated AhR xenobiotic responsive element driven by a firefly luciferase reporter plasmid, pGudLuc6.167. Cells were plated in MEMA (Gibco), supplemented with 10% fetal serum bovine and 1% penicillin-streptomycin solution, in 24-well plate and stimulated with different concentrations of either 3-IAld or 1ME3CA. Luciferase activity, calculated as relative light units (RLU) per microgram of protein and expressed as fold induction, was carried at 2 or 24 hours of exposure. The results clearly showed that 1ME3CA was a more potent inducer of luciferase activity that 3-IAld, as observed by the higher RLU at 2 h and lasting until 24h at the 100 mM concentration (FIG. 21).

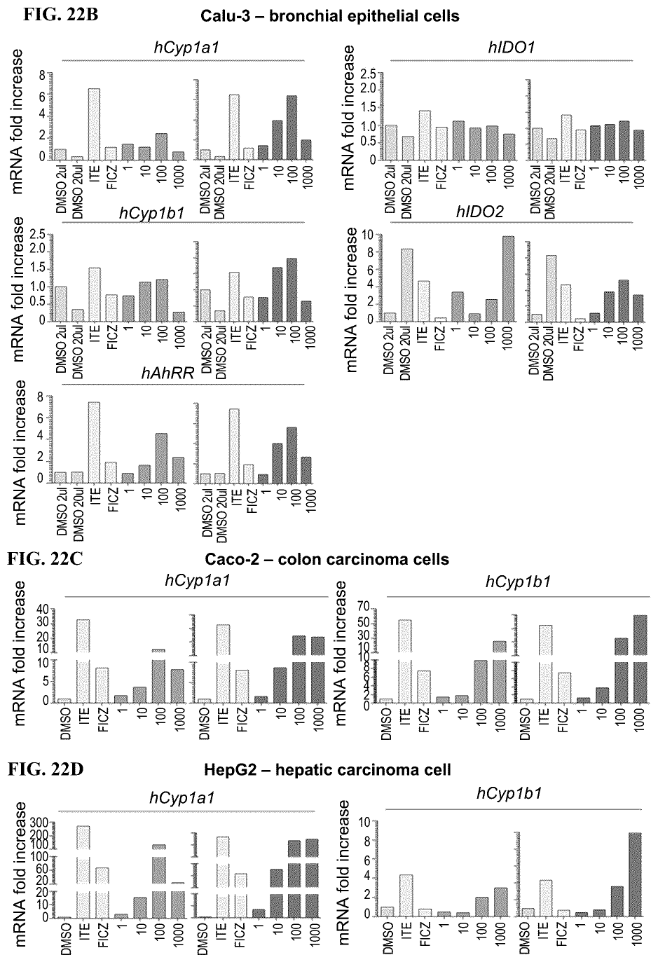

To further characterize the AhR agonistic potential of 1ME3CA, the ability of 3-IAld and 1ME3CA to activate target genes downstream AhR (Cyp1a1, Cyp2a1 and AhRR) in the A549 cell line derived from adenocarcinomic human alveolar basal epithelial cells, in the Calu-3 cell line derived from adenocarcinomic human bronchial epithelial cells, in the CaCo-2 cell line derived from human colon carcinoma and in the HepG32 human liver cancer cell line was comparatively assessed. Cells were exposed to 1, 10, 100 and 1000 M of either molecules or DMSO for 4 hours or overnight (on) at 37° C. before being assessed for the expression of the above-mentioned genes by RT-PCR. The results showed that: i) 1ME3CA promoted the expression of AhR-dependent Cyp1a1, Cyp2a1 and AhRR genes in the cell lines tested to an extent comparable, or even superior, to the reference AhR ligands ITE or FICZ as well as to 3-IAld at a similar concentration, i.e. 100 μM; ii) both molecules acted within the optimal of range concentration of 10-100 μM; inconsistent results were obtained with the higher 1000 M concentration, being the AhR activity increased in the CaCo-2 and the HepG32 cell lines but not on Calu-3 cells; iii) this activity occurred as early as 4 hours of exposure and seemed to maintain thereafter (FIG. 22A-D). No activity was observed on the expression of the IDO1 and IDO2 genes that were assessed for their ability to activate AhR. All together, these data suggest that the AhR agonistic activity of 1ME3CA is comparable, if not superior to that of 3-IAld.

### Pharmacodynamics—Dose Dependent In Vivo Activity

In the DSS+anti-CTLA-4-induced colitis, mice were treated intragastrically (Eudragit formulations) every other day starting 4 days before DSS treatment and continuing until mice sacrifice. Mice were evaluated for disease activity in terms of histopathology, parameters of barrier permeability function and intestinal inflammation.

Assessment of scaling doses of 0.09-0.045-0.022 mg/mouse (corresponding to 4.5, 2.25 and 1.12 mg/kg) in the murine model of immune-mediated colitis (DSS+anti-CTLA-4) was performed as shown in Table 2 below.

TABLE 2
Colitis/intestinal inflammation

| | | 1-methylindole-3- |
|---|---|---|
| | | carboxylic acid |
| | αCTLA-4 | 0.09-0.045-0.022 |
| | 100 μg/mouse | mg/mouse |
| Mice | (Twice per week) | (every other day) |
| 4 C57BL/6 | − | − |
| 4 C57BL/6 | + | − |
| 12 C57BL/6 | + | + |

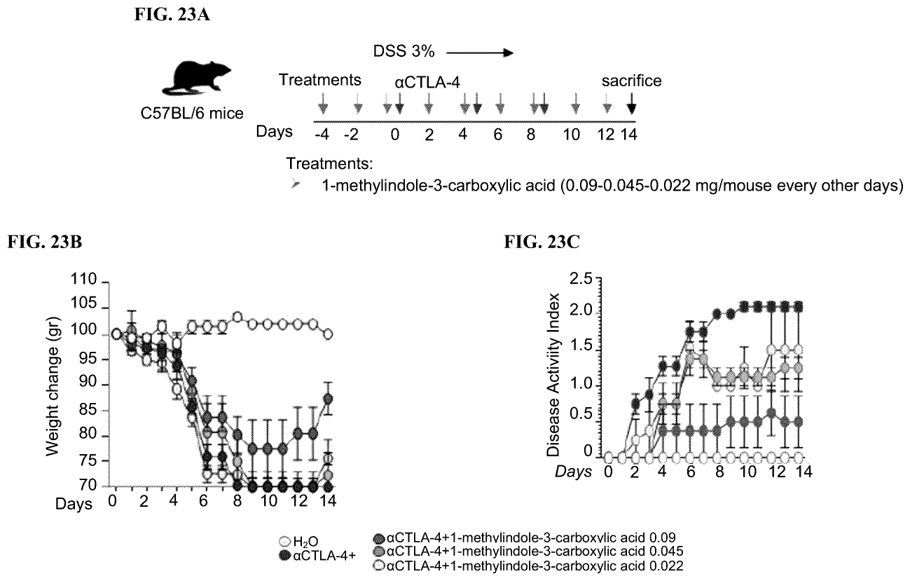

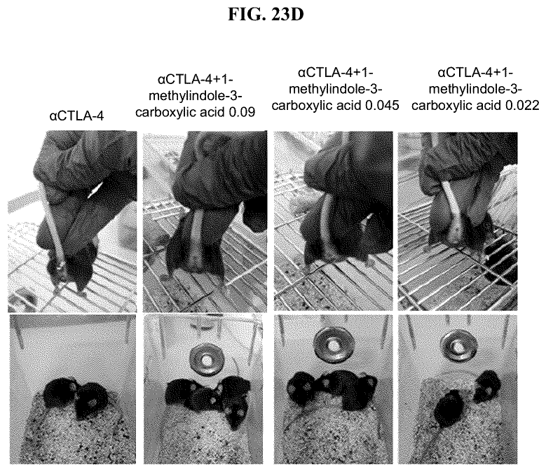

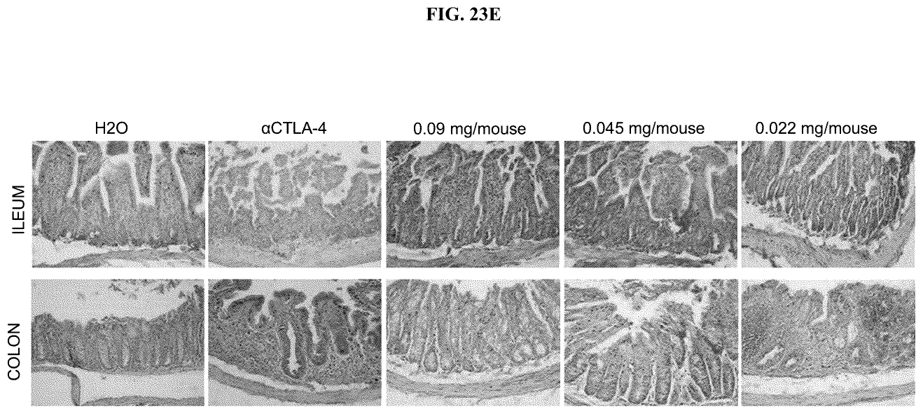

C57BL/6 mice were treated with DSS in drinking water for 1 week followed by a recovery period of another week and administered 100 g of anti-CTLA-4 mAb and 1ME3CA as depicted in the experimental schedule in FIG. 23A. Mice were evaluated for weight change (FIG. 23B), disease activity index (FIG. 23C), clinical morbidity and rectal bleeding (FIG. 23D), colon and ileum histology (FIG. 23E) (PAS staining), ×40 magnification (scale bars, 100 m). Each in vivo experiment includes four to six mice per group (20 mice in each experiment).

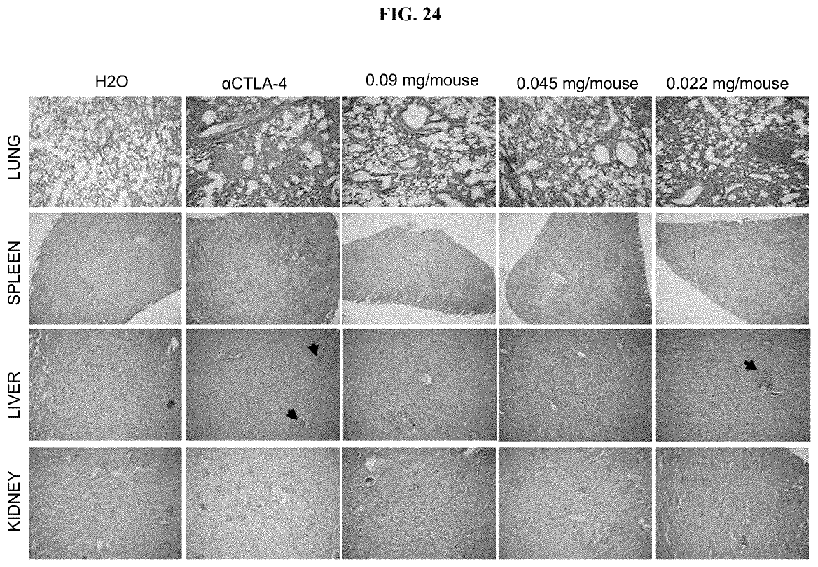

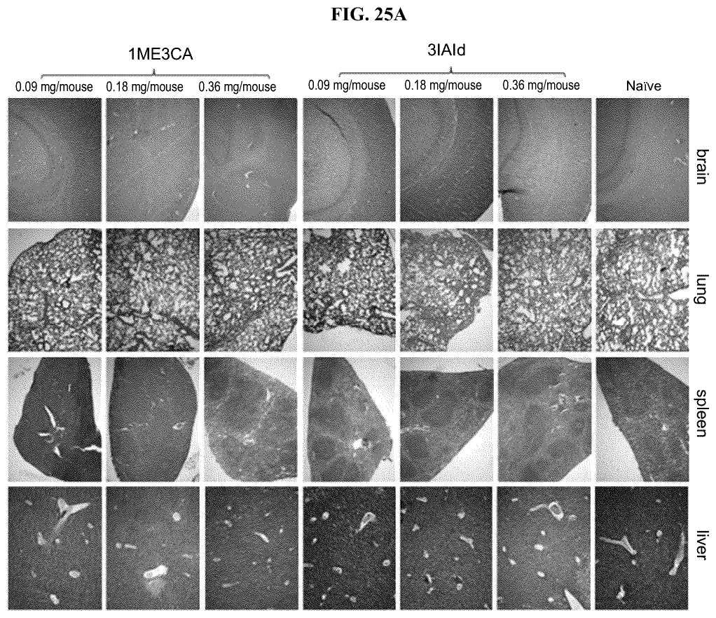

To confirm the effect of 1ME3CA on lymphocytic infiltration, mice were treated as before and assessed for histological changes (PAS staining). Photographs were taken with a high-resolution microscope, lung and spleen ×10 magnification (scale bars, 400 m) and liver and kidney ×20 magnification (scale bars, 200 m). As shown in FIG. 24, 1ME3CA reduces lymphocytic infiltration in anti-CTLA-4-treated mice in a dose-dependent manner with the efficacy dose for 3-IAld being 18 mg/kg and for 1ME3CA being 2.25 mg/kg.

### Toxicology

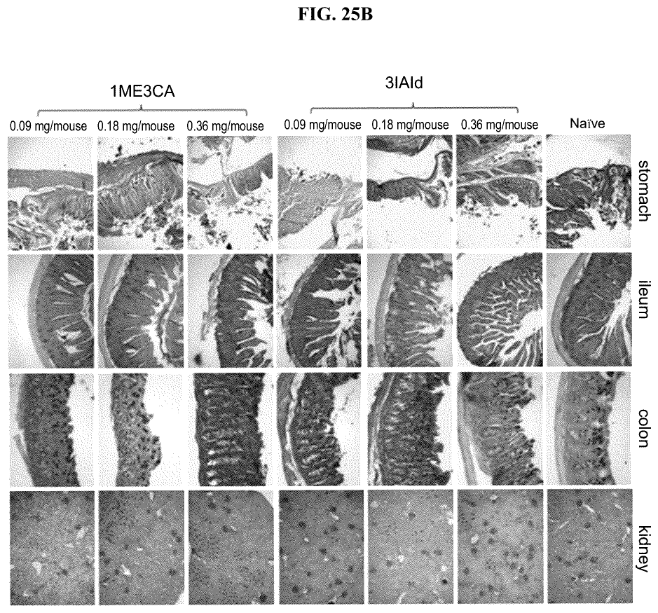

For assess toxicology, C57BL/6 mice received intragastrically escalating doses of 0.36-0.18-0.09 mg/mouse of 3-IAld or 1ME3CA formulated in enteric microparticles for up to 3 weeks as shown in Table 3 below. At the end of treatment, mice were sacrificed, and tissue pathology blindly examined on different organs after PAS staining of different section from each organ. Sections of organs from 4 mice in each group were separately evaluated. The results (FIGS. 25A and 25B) show the absence of visible tissue pathology in all the organs examined at the 3 different concentrations.

TABLE 3
Toxicology

| | 1-methylindole-3- | |
|---|---|---|
| | carboxylic acid | 3-IAld |
| | 0.36-0.18-0.09 | 0.36-0.18-0.09 |
| | mg/mouse | mg/mouse |
| Mice | (every other day) | (every other day) |
| 4 C57BL/6 | − | − |
| 12 C57BL/6 | − | + |
| 12 C57BL/6 | + | − |

### REFERENCES

Alexeev E E, Lanis J M, Kao D J, et al. Microbiota-Derived indole metabolites promote human and murine intestinal homeostasis through regulation of interleukin-10 receptor. Am J Pathol 2018; 188:1183-94.
Andrews M C, Duong C P M, Gopalakrishnan V, Iebba V, Chen W S, Derosa L, et al. Gut microbiota signatures are associated with toxicity to combined CTLA-4 and PD-1 blockade. Nat Med. 2021 August; 27(8):1432-41. doi: 10.1038/s41591-021-01406-6.
Attia P, et al. Autoimmunity correlates with tumor regression in patients with metastatic melanoma treated with anti-cytotoxic T-lymphocyte antigen-4. J Clin Oncol. 2005; 23(25):6043-53. doi:10.1200/JCO.2005.06.205.
Ayrignac X, Goulabchand R, Jeziorski E, et al. Two neurologic facets of CTLA4-related haploinsufficiency. Neurol Neuroimmunol Neuroinflamm. 2020; 7(4): e751.
Bakacs T, Mehrishi J N. Anti-CTLA-4 therapy may have mechanisms similar to those occurring in inherited human CTLA4 haploinsufficiency. Immunobiology. 2015; 220(5):624-5.
Bakhtiar S, Gimez-Diaz L, Jarisch A, Soerensen J, Grimbacher B, Belohradsky B, Keller K M, Rietschel C, Klingebiel T, Koletzko S, Albert M H, Bader P (2017) Treatment of Infantile Inflammatory Bowel Disease and Autoimmunity by Allogeneic Stem Cell Transplantation in LPS-Responsive Beige-Like Anchor Deficiency. Frontiers in immunology 8:52.
Berg D J, Davidson N, Kuhn R, et al. Enterocolitis and colon cancer in interleukin-10-deficient mice are associated with aberrant cytokine production and CD4(+) TH1-like responses. J Clin Invest. 1996; 98: 1010-1020.
Besnard C, Levy E, Aladjidi N, Stolzenberg M C, et al. Pediatric-onset Evans syndrome: Heterogeneous presentation and high frequency of monogenic disorders including LRBA and CTLA4 mutations. Clin Immunol. 2018; 188:52-57. doi: 10.1016/j.clim.2017.12.009.
Borghi M, et al. Targeting the Aryl Hydrocarbon Receptor with Indole-3-Aldehyde Protects from Vulvovaginal Candidiasis via the IL-22-IL-18 Cross-Talk. Front Immunol. 2019; 10:2364.
Bratanič N, Kovač J, Pohar K, Trebusak Podkrajsek K, Ihan A, Battelino T, Avbelj Stefanija M. Multifocal gastric adenocarcinoma in a patient with LRBA deficiency. Orphanet J Rare Dis. 2017 Jul. 18; 12(1):131.
Choi J, Lee S Y. Clinical Characteristics and Treatment of Immune-Related Adverse Events of Immune Checkpoint Inhibitors. Immune Netw. 2020; 20(1):e9.
Compare D, Rocco A, Nardone G. Risk factors in gastric cancer. Eur Rev Med Pharmacol Sci. 2010; 14(4):302-8.
Constant B D, Dutmer C M, Arnold M A, Hall C, Abbott J K, de Zoeten E. Cytotoxic T-Lymphocyte-Associated Antigen 4 Haploinsufficiency Mimics Difficult-to-Treat Inflammatory Bowel Disease. Clinical Gastroenterology and Hepatology 2022; 20: e696-e702.
Cunningham-Rundles C, Bodian C. Common variable immunodeficiency: clinical and immunological features of 248 patients. Clin Immunol. July 1999; 92(1):34-48. doi:10.1006/clim.1999.4725.
Dai X, Zhu B T. Indoleamine 2,3-dioxygenase tissue distribution and cellular localization in mice: implications for its biological functions. J Histochem Cytochem. 2010; 58(1):17-28.
Descamps H C, Herrmann B, Wiredu D, Thaiss C A. The path toward using microbial metabolites as therapies. EBioMedicine. 2019; 44:747-754.
Dhalla F, da Silva S P, Lucas M, Travis S, Chapel H. Review of gastric cancer risk factors in patients with common variable immunodeficiency disorders, resulting in a proposal for a surveillance programme. Clin Exp Immunol. 2011; 165(1):1-7.
Dhar P, McAuley J. The role of the cell surface mucin MUC1 as a barrier to infection and regulator of inflammation. Front Cell Infect Microbiol 2019; 9:117.
Egg D, et al. Increased Risk for Malignancies in 131 Affected CTLA4 Mutation Carriers. Front Immunol. 2018; 9:2012.
Egg D, et al. Therapeutic options for CTLA-4 insufficiency. J Allergy Clin Immunol. 2021; S0091-6749(21)00891-5.
Esser C, Rannug A. The aryl hydrocarbon receptor in barrier organ physiology, immunology, and toxicology. Pharmacol Rev. 2015; 67(2):259-79.
Gimez-Diaz L, August D, Stepensky P, Revel-Vilk S, Seidel M G, Noriko M, Morio T, Worth AJJ, Blessing J, Van de Veerdonk F, Feuchtinger T, Kanariou M, Schmitt-Graeff A, Jung S, Seneviratne S, Burns S, Belohradsky B H, Rezaei N, Bakhtiar S, Speckmann C, Jordan M, Grimbacher B (2016) The extended phenotype of LPS-responsive beige-like anchor protein (LRBA) deficiency. The Journal of allergy and clinical immunology 137 (1):223-230.
Garcia-Perez J E, Baxter R M, Kong D S, Tobin R, McCarter M, Routes J M, Verbsky J, Jordan M B, Dutmer C M, Hsieh EWY. CTLA4 Message Reflects Pathway Disruption in Monogenic Disorders and Under Therapeutic Blockade. Front Immunol. 2019; 10:998.
Gomes-Santos A C, Garcias Moreira T, Barbosa Castro-Junior A, Coelho Horta B, Lemos L, Nogueira Cruz D, Freitas Guimaraes M A, Cara D M, McCafferty D-M, and Caetano Fari A M. New Insights into the Immunological Changes in IL-10-Deficient Mice during the Course of Spontaneous Inflammation in the Gut Mucosa. Clin Dev Immunol 2012; 2012:560817.
Hashimoto T, et al. ACE2 links amino acid malnutrition to microbial ecology and intestinal inflammation. Nature. Jul. 25 2012; 487(7408):477-81.
He Y, Li X, Yu H, Ge Y, Liu Y, Qin X, Jiang M, Wang X. The Functional Role of Fecal Microbiota Transplantation on Dextran Sulfate Sodium-Induced Colitis in Mice. Front Cell Infect Microbiol. 2019; 9:393.
Hodi F S, et al. Improved survival with ipilimumab in patients with metastatic melanoma. N Engl J Med. 2010; 363(8):711-23.
Hoshino A, Toyofuku E, Mitsuiki N, et al. Clinical Courses of IKAROS and CTLA4 Deficiencies: A Systematic Literature Review and Retrospective Longitudinal Study. Front Immunol. 2022; 12:784901.
Hubbard T D, Murray I A, Bisson W H, Lahoti T S, Gowda K, Amin S G, Patterson A D, Perdew G H. Adaptation of the human aryl hydrocarbon receptor to sense microbiota-derived indoles. Sci Rep. 2015; 5:12689. (Hubbard et al 2015 a).
Hubbard T D, Murray I A, Perdew G H. Indole and Tryptophan Metabolism: Endogenous and Dietary Routes to Ah Receptor Activation. Drug Metab Dispos. 2015; 43(10):1522-35. (Hubbard et al 2015 b).
Iida N, Dzutsev A, Stewart C A, Smith L, Bouladoux N, Weingarten R A, et al. Commensal bacteria control cancer response to therapy by modulating the tumor microenvironment. Science. 2013; 342(6161):967-70.
Jamee M, Hosseinzadeh S, Sharifinejad N, Zaki-Dizaji M, Matloubi M, Hasani M, Baris S, Alsabbagh M, Lo B, Azizi G. Comprehensive comparison between 222 CTLA-4 haploinsufficiency and 212 LRBA deficiency patients: a systematic review. Clin Exp Immunol. 2021; 205(1):28-43.
Karamchandani D M, Chetty R. Immune checkpoint inhibitor-induced gastrointestinal and hepatic injury: pathologists' perspective. J Clin Pathol. 2018; 71(8):665-671.
Keubler L M, Buettner M, Hager C, Bleich A. A Multihit Model: Colitis Lessons from the Interleukin-10-deficient Mouse. Inflamm Bowel Dis 2015; 21:1967-1975.
Kiesler P, Fuss I J, Strober W. Experimental Models of Inflammatory Bowel Diseases. Cell Mol Gastroenterol Hepatol. 2015 Mar. 1; 1(2):154-170.
Konopelski P, Ufnal M. Indoles—Gut Bacteria Metabolites of Tryptophan with Pharmacotherapeutic Potential. Curr Drug Metab. 2018; 19(10):883-890.
Kuehn H S, et al. Immune dysregulation in human subjects with heterozygous germline mutations in CTLA4. Science. 2014; 345(6204):1623-1627.
Kumar K K, Burgess A W, Gulbis J M. Structure and function of LGR5: an enigmatic G-protein coupled receptor marking stem cells Protein Sci 2014; 23(5):551-65.
Lamas B, et al. CARD9 impacts colitis by altering gut microbiota metabolism of tryptophan into aryl hydrocarbon receptor ligands. Nat Med. 2016; 22(6):598-605.
Lanz A L, Riester M, Peters P, Schwerd T, et al. Abatacept for treatment-refractory pediatric CTLA4-haploinsufficiency. Clin Immunol. 2021; 229:108779.
Lee J S, et al. AHR drives the development of gut ILC22 cells and postnatal lymphoid tissues via pathways dependent on and independent of Notch. Nat Immunol. 2011; 13(2):144-51.
Lévy E, Stolzenberg M C, Bruneau J, Breton S, Neven B, Sauvion S, Zarhrate M, Nitschké P, Fischer A, Magerus-Chatinet A, Quartier P, Rieux-Laucat F. LRBA deficiency with autoimmunity and early onset chronic erosive polyarthritis. Clin Immunol. 2016 July; 168:88-93.
Lo B, Fritz J M, Su H C, Uzel G, Jordan M B, Lenardo M J. CHAI and LATAIE: new genetic diseases of CTLA-4 checkpoint insufficiency. Blood. 2016 Aug. 25; 128(8):1037-42.
Manicassamy S, Manoharan I. Mouse models of acute and chronic colitis. Methods Mol Biol. 2014; 1194:437-48.
Marin-Acevedo J A, Dholaria B, Soyano A E, Knutson K L, Chumsri S, Lou Y. Next generation of immune checkpoint therapy in cancer: new developments and challenges. J Hematol Oncol. 2018; 11(1):39.
Moraes-Fontes M F, Hsu A P, Caramalho I, Martins C, Aranjo A C, Lourengo F, Taulaigo A V, Lladó A, Holland S M, Uzel G. Fatal CTLA-4 heterozygosity with autoimmunity and recurrent infections: a de novo mutation. Clin Case Rep. 2017; 5(12):2066-2070.
Opitz C A, et al. An endogenous tumour-promoting ligand of the human aryl hydrocarbon receptor. Nature. 2011; 478(7368):197-203.
Perez-Ruiz E, et al. Prophylactic TNF blockade uncouples efficacy and toxicity in dual CTLA-4 and PD-1 immunotherapy. Nature. 2019; 569(7756):428-432.
Puccetti M, Giovagnoli S, Zelante T, Romani L, Ricci M. Development of Novel Indole-3-Aldehyde-Loaded Gastro-Resistant Spray-Dried Microparticles for Postbiotic Small Intestine Local Delivery. J Pharm Sci. 2018; 107(9):2341-2353.
Puccetti M, Pariano M, Borghi M, Barola C, Moretti S, Galarini R, Mosci P, Ricci M, Costantini C, Giovagnoli S. Enteric formulated indole-3-carboxaldehyde targets the aryl hydrocarbon receptor for protection in a murine model of metabolic syndrome. Int J Pharm. 2021; 602:120610.
Qiu J, et al. The aryl hydrocarbon receptor regulates gut immunity through modulation of innate lymphoid cells. Immunity. 2012; 36(1): 92-104.
Renga G, Bellet M M, Pariano M, Gargaro M, Stincardini C, D'Onofrio F, et al. Thymosin alpha₁ protects from CTLA-4 intestinal immunopathology. Life Sci Alliance. 2020; 3(10).
Renga G, Nunzi E, Pariano M, Puccetti M, Bellet M M, Pieraccini G, D'Onofrio F, et al. Optimizing therapeutic outcomes of immune checkpoint blockade by a microbial tryptophan metabolite. J Immunother Cancer. 2022; (3): e003725.
Roager H M, Licht T R. Microbial tryptophan catabolites in health and disease. Nat Commun. 2018; 9(1):3294.
Safe S, Jayaraman A, Chapkin R S. Ah receptor ligands and their impacts on gut resilience: structure-activity effects. Crit Rev Toxicol. 2020; 50(6):463-473.
Salavoura K, Kolialexi A, Tsangaris G, Mavrou A. Development of cancer in patients with primary immunodeficiencies. Anticancer Res. 2008; 28(2B):1263-9.
Schoenfeld A J, Hellmann M D. Acquired Resistance to Immune Checkpoint Inhibitors. Cancer Cell. 2020; 37(4):443-55.
Schubert D, et al. Autosomal dominant immune dysregulation syndrome in humans with CTLA4 mutations. Nat Med. 2014; 20(12):1410-1416.
Schwab C, Gabrysch A, Olbrich P, et al. Phenotype, penetrance, and treatment of 133 cytotoxic T-lymphocyte antigen 4-insufficient subjects. J Allergy Clin Immunol. 2018; 142(6):1932-1946. doi: 10.1016/j.jaci.2018.02.055.
Scott S A, Fu J, Chang P V. Microbial tryptophan metabolites regulate gut barrier function via the aryl hydrocarbon receptor. Proc Natl Acad Sci USA. 2020; 117(32):19376-19387. doi:10.1073/pnas.2000047117.
Semo Oz R, M S T (2019) Arthritis in children with LRBA deficiency—case report and literature review. Pediatric rheumatology online journal 17 (1):82.
Shimada Y, et al. Commensal bacteria-dependent indole production enhances epithelial barrier function in the colon. PLoS One. 2013; 8(11): e80604.
Soler-Palacin P, Garcia-Prat M, Martin-Nalda A, Franco-Jarava C, Riviere J G, Plaja A, Bezdan D, Bosio M, Martinez-Gallo M, Ossowski S, Colobran R (2018) LRBA Deficiency in a Patient With a Novel Homozygous Mutation Due to Chromosome 4 Segmental Uniparental Isodisomy. Frontiers in immunology 9:2397.
Spits H, et al. Innate lymphoid cells—a proposal for uniform nomenclature. Nat Rev Immunol. 2013; 13(2):145-9.
Stockinger B, Di Meglio P, Gialitakis M, Duarte J H. The aryl hydrocarbon receptor: multitasking in the immune system. Annu Rev Immunol. 2014; 32:403-32.
Stockinger B, Shah K, Wincent E. AHR in the intertinal microenvironment: safeguarding barrier function. Nat Rev Gastroenterol Hepatol. 2021; 18(8):559-570.
Swimm A, et al. Indoles derived from intestinal microbiota act via type I interferon signaling to limit graft-versus-host disease. Blood. 2018; 132(23):2506-2519. Tesch V K, Abolhassani H, Shadur B, Zobel J, Mareika Y, Sharapova S, Karakoc-Aydiner E, Rivière J G, Garcia-Prat M, Moes N, Haerynck F, Gonzales-Granado L I, Santos Pěrez J L, Mukhina A, Shcherbina A, Aghamohammadi A, Hammarström L, Dogu F, Haskologlu S, Îkincioǧullari A I, Köstel Bal S, Baris S, Kilic S S, Karaca N E, Kutukculer N, Girschick H, Kolios A, Keles S, Uygun V, Stepensky P, Worth A, van Montfrans J M, Peters AMJ, Meyts I, Adeli M, Marzollo A, Padem N, Khojah A M, Chavoshzadeh Z, Avbelj Stefanija M, Bakhtiar S, Florkin B, Meeths M, Gamez L, Grimbacher B, Seppanen MRJ, Lankester A, Gennery A R, Seidel M G (2020) Long-term outcome of LRBA deficiency in 76 patients after various treatment modalities as evaluated by the immune deficiency and dysregulation activity (IDDA) score. The Journal of allergy and clinical immunology 145 (5):1452-1463.
Tesi B, Priftakis P, Lindgren F, Chiang S C, Kartalis N, Lofstedt A, Lörinc E, Henter J I, Winiarski J, Bryceson Y T, Meeths M. Successful Hematopoietic Stem Cell Transplantation in a Patient with LPS-Responsive Beige-Like Anchor (LRBA) Gene Mutation. J Clin Immunol. 2016 July; 36(5):480-9.
Tivol E A, Borriello F, Schweitzer A N, Lynch W P, Bluestone J A, Sharpe A H. Loss of CTLA-4 leads to massive lymphoproliferation and fatal multiorgan tissue destruction, revealing a critical negative regulatory role of CTLA-4. Immunity. 1995; 3(5):541-7.
Van Leeuwen E M, Cuadrado E, Gerrits A M, Witteveen E, de Bree G J. Treatment of Intracerebral Lesions with Abatacept in a CTLA4-Haploinsufficient Patient. J Clin Immunol. 2018; 38(4):464-467.
Wang F, Yin Q, Chen L, Davis M M. Bifidobacterium can mitigate intestinal immunopathology in the context of CTLA-4 blockade. Proc Natl Acad Sci USA. 2018; 115(1):157-161.
Wang T, et al. Probiotics Lactobacillus reuteri Abrogates Immune Checkpoint Blockade-Associated Colitis by Inhibiting Group 3 Innate Lymphoid Cells. Front Immunol. 2019; 10:1235.
Westdorp et al., Mechanisms of Immune Checkpoint Inhibitor-Mediated Colitis Front Immunol. 2021; 12: 768957.
Wikoff W R, et al. Metabolomics analysis reveals large effects of gut microflora on mammalian blood metabolites. Proc Natl Acad Sci USA. 2009; 106(10):3698-703.
Yu X, Wang Y, Deng M, et al. The basic leucine zipper transcription factor NFIL3 directs the development of a common innate lymphoid cell precursor. Elife 2014; 3 10.7554/eLife.04406.
Zelante T, et al. Tryptophan catabolites from microbiota engage aryl hydrocarbon receptor and balance mucosal reactivity via interleukin-22. Immunity. 2013; 39(2):372-85.
Zhang L S, Davies S S. Microbial metabolism of dietary components to bioactive metabolites: opportunities for new therapeutic interventions. Genome Med. 2016; 8(1):46.

1. A method of treating a CTLA-4 checkpoint related immunodeficiency in a patient comprising administering a therapeutically effective amount of a pharmaceutical composition comprising 1H-indole-3-carboxaldehyde (3-IAld) to the patient in need thereof.

2. The method of claim 1, wherein the CTLA-4 checkpoint related immunodeficiency is selected from the group consisting of: CTLA-4 haploinsufficiency with autoimmune infiltration (CHAI), lipopolysaccharide-responsive beige-like anchor (LRBA) deficiency (LATAIE), a regulatory T (Treg) cell defect, autoimmune infiltration, enteropathy disease, gut inflammation, immune-mediated colitis, gastrointestinal disorder, and gastric atrophy.

3. The method of claim 1, wherein the CTLA-4 checkpoint related immunodeficiency is immune-mediated colitis.

4. The method of claim 1, wherein the pharmaceutical composition comprises a pharmaceutically acceptable carrier.

5. The method of claim 4, wherein the pharmaceutically acceptable carrier is at least one polymer.

6. (canceled)

7. The method of claim 6, wherein the set of polymers are Eudragit® polymers.

8. The method of claim 1, wherein the pharmaceutical composition is formulated for enteric delivery or is orally administered.

9. (canceled)

10. The method of claim 1, wherein the pharmaceutical composition is in a form selected from a capsule, tablet, gel tablet, gel capsule, gel, liquid, and gummy.

11. (canceled)

12. The method of claim 1, wherein the pharmaceutical composition is administered at an interval of every other day (q.o.d).

13. The method of claim 1, wherein the pharmaceutical composition is administered at a 3-IAld dosage of at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, least about 16 mg/kg, at least about 17 mg/kg, or least about 18 mg/kg.

14. The method of claim 13, wherein the 3-IAld dosage is about 18 mg/kg.

15. A method of treating a CTLA-4 checkpoint related immunodeficiency in a patient comprising administering a therapeutically effective amount of a pharmaceutical composition comprising 1-methylindole-3-carboxylic acid to the patient in need thereof.

16. The method of claim 15, wherein the CTLA-4 checkpoint related immunodeficiency is selected from the group consisting of: CTLA-4 haploinsufficiency with autoimmune infiltration (CHAI), lipopolysaccharide-responsive beige-like anchor (LRBA) deficiency (LATAIE), a regulatory T (Treg) cell defect, autoimmune infiltration, enteropathy disease, gut inflammation, immune-mediated colitis, gastrointestinal disorder, and gastric atrophy.

17. The method of claim 15, wherein the CTLA-4 checkpoint related immunodeficiency is immune-mediated colitis.

18. The method of claim 15, wherein the pharmaceutical composition comprises a pharmaceutically acceptable carrier.

19. The method of claim 18, wherein the pharmaceutically acceptable carrier is at least one polymer.

20. The method of claim 18, wherein the pharmaceutically acceptable carrier is a set of polymers.

21. (canceled)

22. The method of claim 15, wherein the pharmaceutical composition is formulated for enteric delivery or is orally administered.

23. (canceled)

24. The method of claim 15, wherein the pharmaceutical composition is in a form selected from a capsule, tablet, gel tablet, gel capsule, gel, liquid, and gummy.

25. (canceled)

26. The method of claim 15, wherein the pharmaceutical composition is administered at an interval of every other day (q.o.d).

27. The method of claim 15, wherein the pharmaceutical composition is administered at a 1-methylindole-3-carboxylic acid dosage of at least about 2 mg/kg, at least 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, least about 16 mg/kg, at least about 17 mg/kg, or least about 18 mg/kg.

28. The method of claim 15, wherein the pharmaceutical composition is administered at a 1-methylindole-3-carboxylic acid dosage of about 2.25 mg/kg.
